(19) United States
(12) Patent Application Publication
(10) Pub. No.: US 2017/0126613 A1
(43) Pub. Date: May 4, 2017

(54) INSTANT INFORMATION EXCHANGE SYSTEM AND METHOD FOR ONLINE SPORTS TEAMS

(71) Applicant: JOIIUP TECHNOLOGY INC., Zhubei City (TW)

(72) Inventors: Ling-Syau CHEN, ZHUBEI CITY (TW); Che-Hsien TSAI, ZHUBEI CITY (TW); Jen-Bin KUO, ZHUBEI CITY (TW); Chia-Jung LEE, ZHUBEI CITY (TW); Yuan-Chang CHEN, ZHUBEI CITY (TW); Yen-Cheng CHEN, ZHUBEI CITY (TW); Yu-Hsuan LEE, Kaohsiung City (TW)

(73) Assignee: JOIIUP TECHNOLOGY INC., ZHUBEI CITY (TW)

(21) Appl. No.: 14/931,556

(22) Filed: Nov. 3, 2015

(51) Int. Cl. H04L 12/58; A61B 5/026; A61B 5/01; A61B 5/0488; A63B 24/00; A61B 5/021

(52) U.S. Cl. CPC H04L 51/32; A63B 24/0062; A63B 24/0084; A61B 5/021; A61B 5/01; A61B 5/0488; A61B 5/026; A61B 2503/12

(57) ABSTRACT

An instant information exchange system and method for online sports teams is to include a cloud server having a sports information exchange platform connected with electronic devices via Internet to perform information exchange. The system accepts a user to create sports teams in the sports information exchange platform via the electronic devices and customize sports events and rules to provide a competition mode for the members to perform competition event with teamwork mechanism. The system will receive a physiological value transmitted by the electronic device to be a score as reference. The sports team created by the user can achieve a goal through teamwork and team competition at different time and different locations. The user's sports team can reduce the invitation and waiting time when different sports teams are performing the competition to achieve the exercise purpose and the exercise efficiency.

### BACKGROUND OF THE INVENTION

1. Field of the Invention

The present invention relates to an instant information exchange system and method, and more particularly to an instant information exchange system for online sports teams, and the sports team having many different users at different cities can finish sports events together to compete with different sports teams.

2. Description of Related Art

In recent years, people's health consciousness is awaked and sport and exercise trend prevails. Sport and exercise can release pressure or reduce fatigue and interpersonal relationship can be improved when mood is changing. In daily life, people with sport and exercise habits will have positive effect in self-confidence and self-esteem. Sport and exercise can further enhance metabolism and prevent cardiovascular disease to reduce risk of heart attack and stroke. Sport and exercise can also reduce cholesterol, high blood pressure and so on. Most civilization diseases in modern people relate to persistence or not to exercise. Therefore, sport and exercise not only maintain physical and mental health, but also protect human body. Muscle can be developed at sports to cover bones and joints. Muscle can also support weight and protect important tissues in human body to strengthen human physique.

For modern people, the convenient and common sports and exercise is walking, jogging, treadmill, aerobics, boxing aerobics, cycling, hiking, swimming, etc. Sport and exercise management is usually to calculate exercise time, distance or steps, and the exercise process is recorded by the user himself as reference for future exercising. Individual exercise allows high efficiency and high degree of freedom but is prone to laziness or lack of motivation. If someone wants to recruit people with the same habits to increase motive and fun for exercising, exercise location, contact information and time are required to be arranged specifically, which may lose efficiency and fun. Therefore, some people are bothered because people with the same habits are hard to find. In accordance with the conventional technique, the following methods are tried to solve the aforementioned problems.

For example, a main purpose of a prior art (Taiwan Patent No. I423134: METHOD AND SYSTEM FOR ENHANCING THE INTERACTION LEVEL AMONGST USERS IN A SOCIAL NETWORK APPLICATION) is to interact with friends or virtual users of the friends when users are not online. The interaction level is not only to show user names on the virtual users, but also implements a data digging method to acquire personal information or an interaction information among different virtual users of a first, a second, a third and a fourth virtual user. The personal information and the interaction information are analyzed, and the interaction is performed in accordance with the analyzed personal information and the analyzed interaction information to enhance the interaction level among different users.

Moreover, such as miCoach PACER released by Adidas, SportWatch products with Nike+ Running website platform released by Nike, mySports exercise community website developed by Taiwan Mobile Co., Ltd., free jogging community website developed by WingStyle and Taiwan sports information website (also called iSports) and so on, the purpose of the main design of the aforementioned products is to assist sports and exercises to enhance sports and exercises efficiency of the users. The miCoach and Nike+ can make a sports training plan for individual user and publish a result of the plan on a network platform. The user can see other users' record through the network platform. In addition, the mySports, WingStyle and iSports provide a social network platform for those users who like to exercise, and the social network platform includes functions similar to the social network website. In the social network platform, people with the same sport and exercise habits can be found at one region, and a mutual message function and a message transmitting function on the social network platform can arrange all of the users together to gather at the specific exercise location together. The goal is to exercise together to increase fun and motive for sports.

According to the aforementioned technique, the prior arts are to implement interaction manner between the users in the social network website to raise the interactive level between the different users. Although the method can quickly connect unknown users together to shorten time to find people with the same sport and exercise habits, the exercise cannot be actually and efficiently performed. Especially, the mutual communication happens online, no mutual tie or mutual influences are involved in the execution of the exercise. Furthermore, the aforementioned miCoach or Nike+ includes a promote result by making an exercise training plan and the exercise training plan can be immediately reviewed to remind the user what the goal of the exercise is. However, a long term self-training is prone to lack of will power and tendency to laziness, and the exercise training plan is useless. Moreover, the people with the same sport and exercise habits at the same region can be found in the social network platform, such as mySports, WingStyle, iSports and so on, to be more efficient to gather people with the same sport and exercise habits together to accomplish team sports. Although the fun and motive of the sports can be enhanced, the team sports method to gather together at the specific time and location is inconvenient, inefficient and allowing less freedom for most of the people. Therefore, how to provide the users to include high efficiency and freedom to exercise with plans and fun and how to increase users' sport and exercise times and increase physical health at the same time requires further studies to find a better solution.

### SUMMARY OF THE INVENTION

One objective of the present invention is to provide an instant information exchange system method for online sports teams and the user can create an online sports team. The sports teams can achieve a training plan at different time and different places to reduce waiting time to enhance sports efficiency.

In order to achieve the aforementioned method, the instant information exchange method for the online sports teams is executed in a sports information exchange platform and the method includes the following steps:

accepting a user from a plurality of members to create at least one of the sports teams,

customizing at least one sports event in one of the sports teams created by the user;

adding at least one of the members in one of the sports teams created by the user;

providing a competition mode for the members to perform the at least one competition event with teamwork mechanism; and

providing the user to review one of the sports teams created by the user and instant status of the one of the sports teams.

The aforementioned method is to create new sports team for the user in the sports information exchange platform and the user can edit and customize sports events and rules, such as a condition to finish the sports event, a score counting method of the sports event, of the sports team in the sports information exchange platform. The proposer (the captain) of the sports team can let other users join the members to be the teammates by an invitation method, an accepting method and so on. The sports information exchange platform also provides a competition mode to let the sports team and other sports teams perform the competition event with teamwork mechanism. The teammates of the sports team must cooperate and remind each other to finish a sectional or a complete competition event in a sequential order, a relaying order or any other orders. During the competition, the sports information exchange platform further provides the user to review the sports team and instant status of the sports team to improve the efficiency of the sports team.

In order to achieve the aforementioned projective, the instant information exchange system for online sports teams includes a cloud server and the cloud server includes:

a network module;

a memory unit;

a display;

an input module; and

a processor connected with the network module, the memory unit, the display and the input module;

wherein the electronic device is to show the add-new-team interface, the team-contest-customizing interface and the team-managing interface on the display, operate from the input module by the user and add the new sports teams to perform the at least one competition event with teamwork mechanism.

### BRIEF DESCRIPTION OF THE DRAWINGS

FIG. 1 is an external view of an instant information exchange system in a preferred embodiment of the present invention;

FIG. 2 is a block diagram of the electronic device in the preferred embodiment of the present invention;

FIG. 3 is a page view of a sports information exchange platform in the preferred embodiment of the present invention;

FIG. 4 is a view of the add-new-team page in the preferred embodiment of the present invention;

FIG. 5 is a view of an invitation page in the add-new-team interface in the preferred embodiment of the present invention;

FIG. 6 is a view of an invitation letter in the add-new-team interface in the preferred embodiment of the present invention;

FIG. 7 is a view of a deletion letter in the add-new-team interface in the preferred embodiment of the present invention;

FIG. 8 is a page view of another sports information exchange platform in the preferred embodiment of the present invention;

FIG. 9 is a view of a detailed information page for the sports team in preparation in the preferred embodiment of the present invention;

FIG. 10 is a view of the detailed information page for the sports team in progress in the preferred embodiment of the present invention;

FIG. 11 is a view of a competition score page for the sports team in progress in the preferred embodiment of the present invention;

FIG. 12 is a view of an end status page in the preferred embodiment of the present invention;

FIG. 13 is a view of a detailed information page of the end status in the preferred embodiment of the present invention;

FIG. 14 is a view of a competition score page of the end status in the preferred embodiment of the present invention;

FIG. 15 is a view of a restart page of the end status in the preferred embodiment of the present invention;

FIG. 16 is a view of the sports information exchange platform page in the preferred embodiment of the present invention;

FIG. 17 is a view of a team contest mode page in the preferred embodiment of the present invention;

FIG. 18 is a view of the invitation page of the team contest mode in the preferred embodiment of the present invention;

FIG. 19 is a view of the invitation letter of the team contest mode in the preferred embodiment of the present invention;

FIG. 20 is a view of the competition score page of the team contest mode in the preferred embodiment of the present invention;

FIG. 21 is a view of another sports information exchange platform page in the preferred embodiment of the present invention;

FIG. 22 is a view of still another sports information exchange platform page in the preferred embodiment of the present invention;

FIG. 23 is a view of the competition score page of further another sports team in the preferred embodiment of the present invention;

FIG. 24 is a flow chart of the instant information exchange method in the preferred embodiment of the present invention;

FIG. 25 is a flow chart of subscriber verification in the preferred embodiment of the present invention;

FIG. 26 is a flow chart of customizing sports events and rules in the preferred embodiment of the present invention;

FIG. 27 is a flow chart of editing information in the detailed information page in the preferred embodiment of the present invention;

FIG. 28A and FIG. 28B are flow charts of establishing the team contest mode and a teamwork mechanism in the preferred embodiment of the present invention.

### DETAILED DESCRIPTION OF THE PREFERRED EMBODIMENT

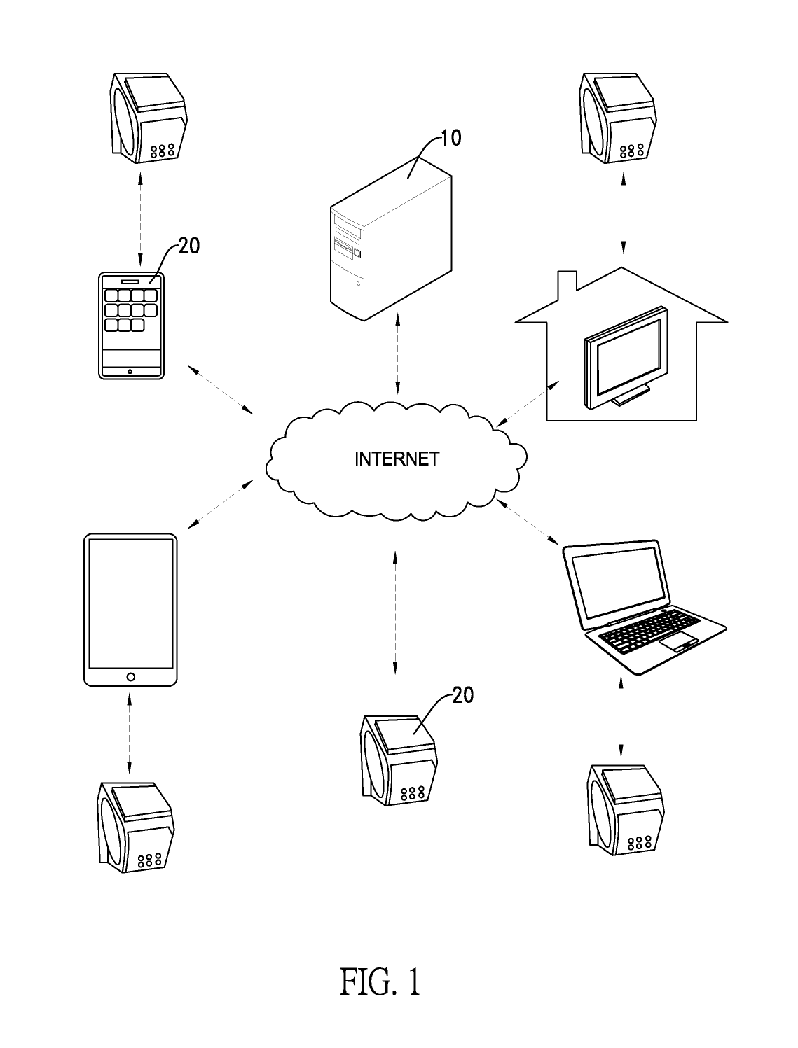

With reference to FIG. 1 regarding an instant information exchange system for online sports team in a preferred embodiment of the present invention, the instant information exchange system includes a cloud server 10 to provide a sports information exchange platform. The sports information exchange platform includes an add-new-team interface, a team-contest-customizing interface, and a team managing interface and information exchange is performed directly or indirectly by network and at least one electronic device 20. Users at different regions can start or join a sports team on the sports information exchange platform by the at least one electronic device 20. In the present embodiment, the sports information exchange platform can support multiple electronic devices 20 connecting at the same time and the electronic device 20 can be an electronic product, such as a digital TV, a tablet, a smart phone, a laptop, a desktop, a wearable computing device and so on. Moreover, when the user carries a wearable computing device during exercise, the wearable computing device senses physiological status information of the user and immediately transmits the physiological status information to the cloud server 10 via the network. In addition, the wearable computing device can store the physiological status information of the user firstly and transmit the physiological status information of the user to the cloud server 10 by other electronic devices 20 (such as digital TV, tablet, smart phone, laptop and desktop) after exercising. The physiological status information of the user can be stored in other electronic devices 20 for the user to record all of the physiological status information. It should be noted that the aforementioned electronic devices are used as example, and it is not to limit only those electronic devices can be used in the present invention.

The aforementioned sports information exchange platform is used for a user to create at least one sports team on the add-new-team interface and configure sports events and rules on the add-new-team interface. The user is a proposer of the sports team and the user can utilize the team-contest-customizing interface to let at least two members in the sports team or at least one member in the sports team and at least one member from different sports team to join a team contest mode, and at least one competition event with teamwork mechanism is performed and valid sports scores of the sports team is calculated. In addition, during the competitions of the sports teams, the user can review the sports team, which the user created, and the instant status of the sports team from the team-managing interface. The teamwork mechanism described in the present invention is an interactive manner (such as all of the members working together, team contest and cooperation between teams), and the members in the sports team have to work together or remind each other to achieve one goal or a training project.

It should be noted that the sports team in the present invention can include one person or many people. Therefore, more than two users can respectively be the proposer in different sports teams, and more than two users can join in the same one sports team to be the members. The sports teams can respectively join at least one competition with teamwork mechanism and valid sports scores at the sports teams can be calculated respectively. In addition, all of the members in the sports teams can also enter the team contest mode to join the at least one competition with teamwork mechanism. Moreover, the user who has been the proposer or the member in the sports team can also create another new sports team and invite other proposers or members from other sports teams to join the new team. Therefore, when only one person is in the sports team, the valid sports score of the person is calculated. When many people are in the sports team, the valid sports scores of the people are calculated.

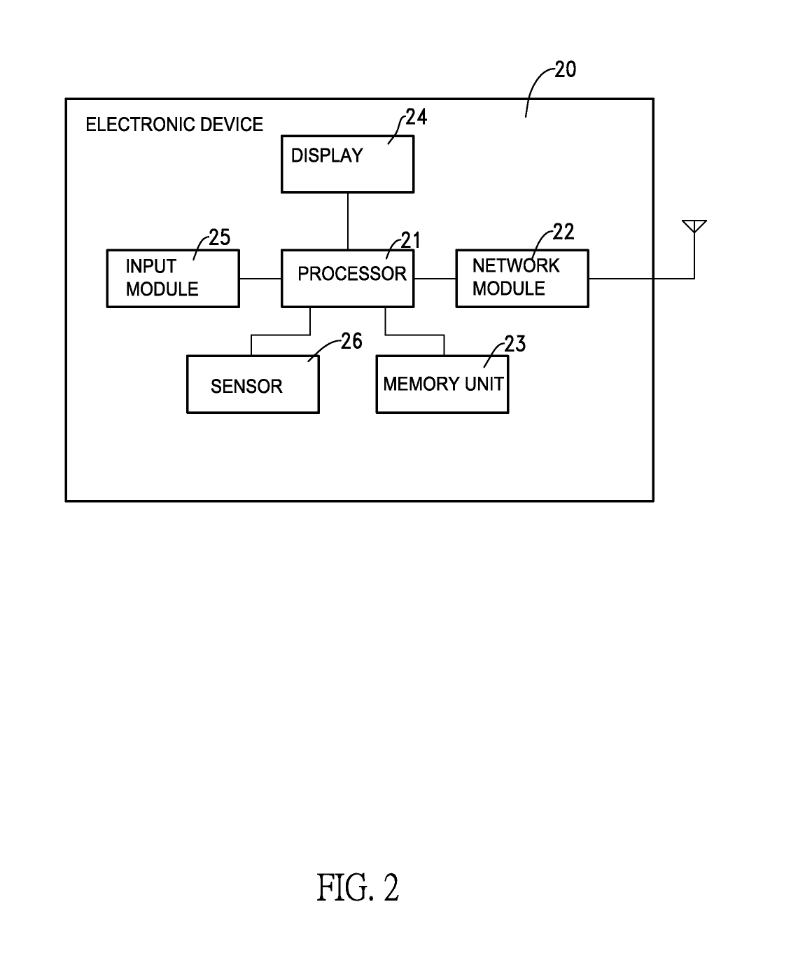

With reference to FIG. 2, a structure of an electronic device 20 in the embodiment is described, and the electronic device 20 includes a processor 21, a network module 22, a memory unit 23, a display 24 and an input module 25. The processor 21 is respectively connected to the network module 22, the memory unit 23, the display 24 and the input module 25. The network module 22 supports one network protocol to connect the electronic device 20 with the cloud server 10 via Internet and receive information from the cloud server 10. Alternatively, the electronic device 20 can perform data exchange indirectly with the cloud server 10 via Internet. The memory unit 23 is to store all of the information received from the network module 22. The processor 21 is to display the information of the sports information exchange platform, such as the add-new-team interface, the team-contest-customizing interface and the team-managing interface for the users to perform the corresponding operation by the input module 25 to create a new team or join a competition. In the present embodiment, the electronic device 20 further includes a sensor 26 electrically connected to the processor 21 and is configured to detect a physiological value of the user, and the sensor 26 can transmit the physiological value directly or indirectly to the cloud server 10. The physiological value can be reference for the value of the competition score. In the present embodiment, the valid sports score can be calculated in the team contest mode and the valid sports score can be the value of the competition score. The physiological value can be information such as heartbeat, heart rate, pulse, blood pressure, blood glucose, body temperature, electromyography (EMG), galvanic skin response (GSR), heart rate variability (HRV), blood flow, etc.

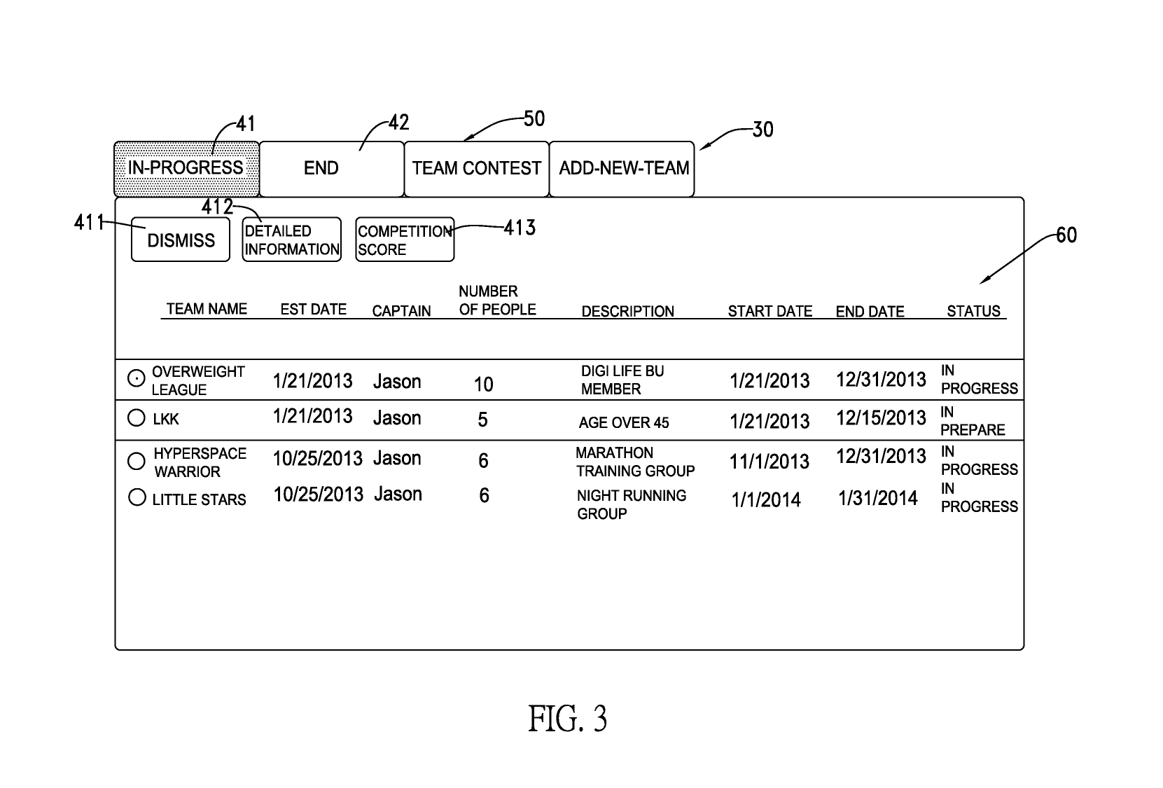

With reference to FIG. 3, the sports information exchange platform in the present embodiment is represented in a webpage as an example, but it is not limited herein. The sports information exchange platform can be used for more than one person to log in. When the user logs in a front page of the sports information exchange platform, multiple interfaces on the front page can be selected to switch from the add-new-team interface 30, the team-managing interface 40 and the team-contest-customizing interface 50.

The team-managing interface 40 includes an in-progress menu 41 and an end menu 42. The in-progress menu 41 is to show the teams, which are still in the competition or are going to start a competition, in a list. As long as the user is the proposer or an invitee and the user joins the team which is in progress or is going to start a competition, a status column 60 will show “in progress”.

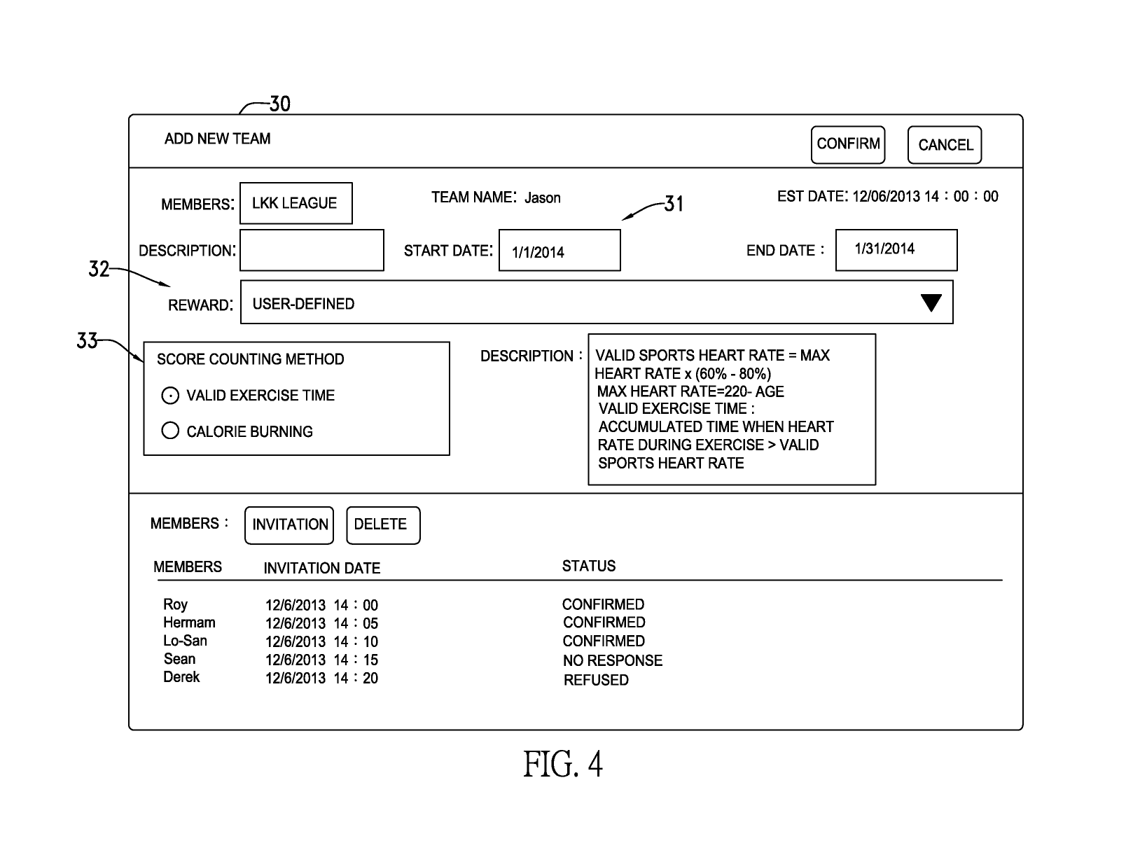

With reference to FIG. 4, the add-new-team interface 30 is to provide the user to initiatively become the proposer (a captain) to recruit a team and include a captain authority, which can invite the members or other proposers to join. The captain is required to firstly create a team name (such as a LKK league and so on) in the add-new-team interface 30 and set a competition start date and a competition end date of the sports team in a competition period column 31. When the competition time is ended, the sports information exchange platform will release the competition result. The add-new-team interface 30 also includes a reward-customizing column 32 for the captain to input a reward or choose one canned text from canned text option provided by the sports information exchange platform (such as congratulations for winners, “cheer up and try next time” for loser and so on). Furthermore, the add-new-team interface 30 also provides a score-counting-method option 33 and sets a score counting pattern from the score-counting-method option 33 to increase interest and excitement in contest. The score counting pattern can be set by the captain to include a valid sports sequencing and a calorie consumption sequencing. The valid sport sequencing is to arrange an ordered sequence in accordance with a valid exercise time and the calorie consumption sequencing is to arrange an ordered sequence in accordance with calorie burning. The valid exercise time relates to the members' ages and physiological value. The calorie burning relates to the members' ages, physiological value, and weights.

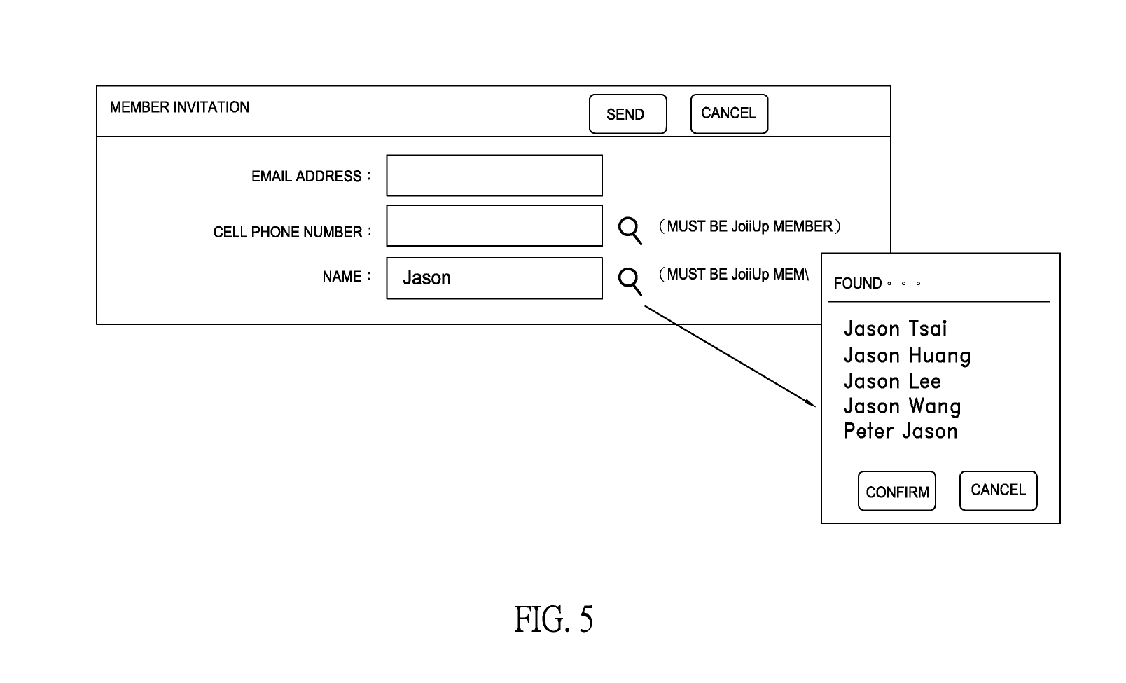

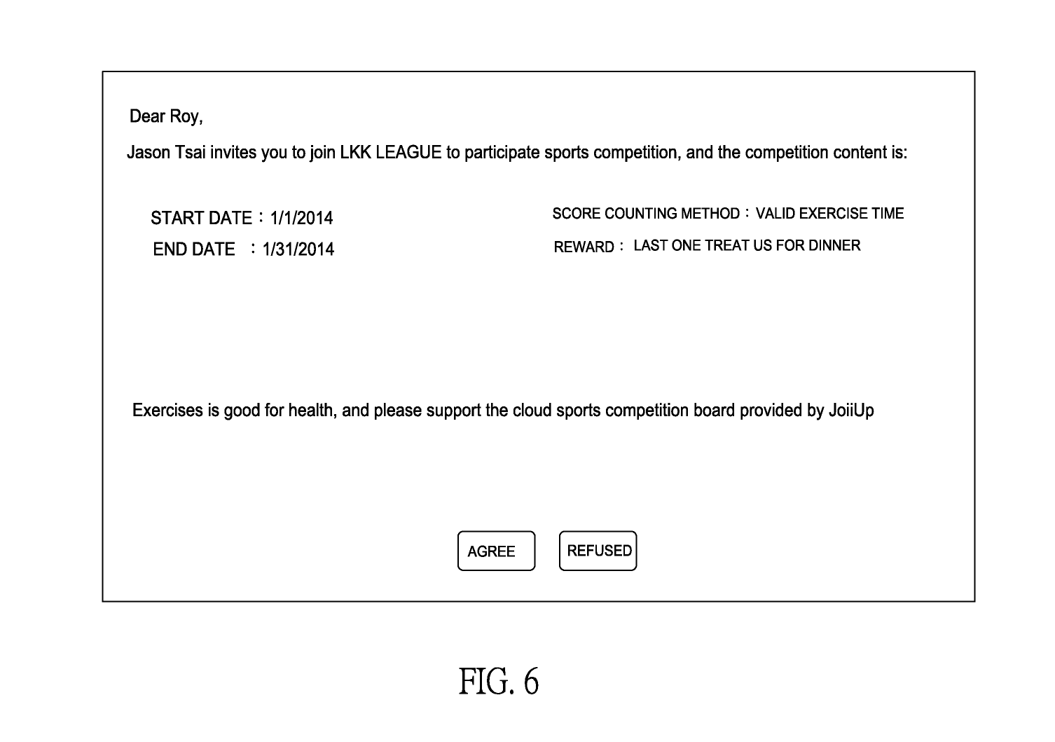

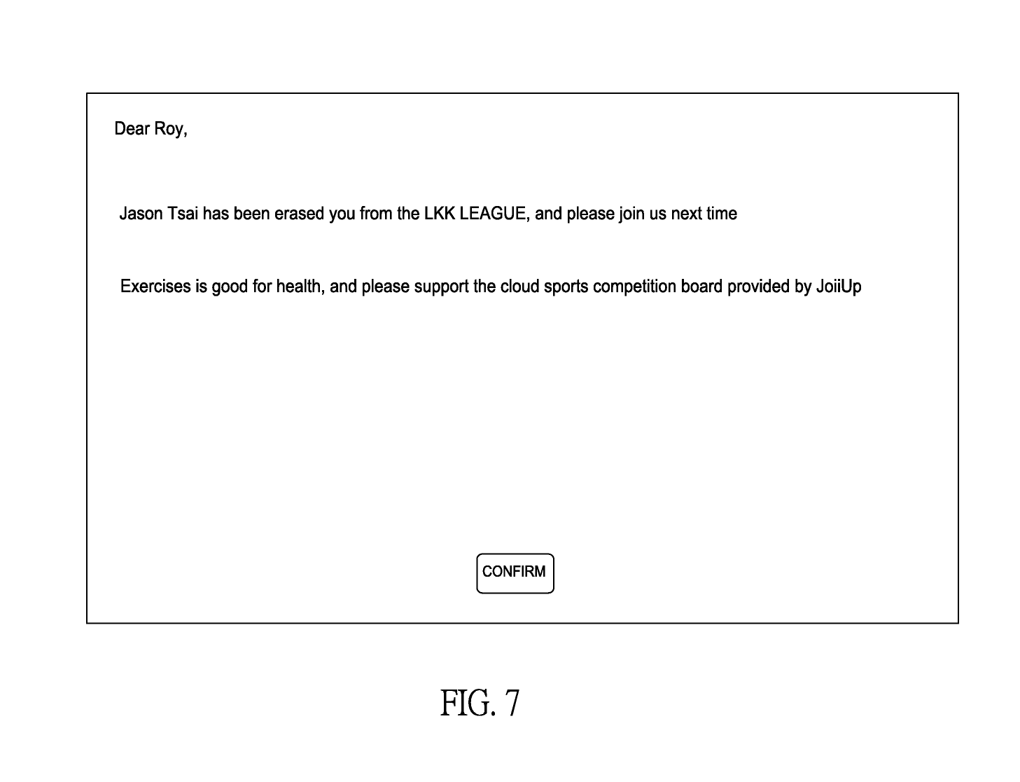

An invitation option and a delete option are set up at the bottom of the add-new-team interface 30. With reference to FIG. 5, when the captain clicks the invitation option, an invitation page 301 for adding a new team is provided. The captain can input new members' email addresses and send the invitations or search for other users' names or cell phone numbers to send the invitation letters. After the invitation letters are sent, with reference to FIG. 6, the sports information exchange platform will send new team invitation messages to specific users. For example, the new team invitation messages are sent to the email addresses of the specific users or the new team invitation messages can be seen when the specific users log in the sports information exchange platform. In addition, the captain can remove any one of the members by the delete option. With reference to FIG. 7, when a specific user is removed, the members will receive a message from a deletion notice letter 303. If the user receiving the invitation letter does not respond, the captain can resend the invitation letter via the invitation option again.

The aforementioned invitees can receive the invitations from emails and log in the sports information exchange platform to respond to the invitations. If the invitees are not valid users, they have to enter a user verify procedure to complete registers.

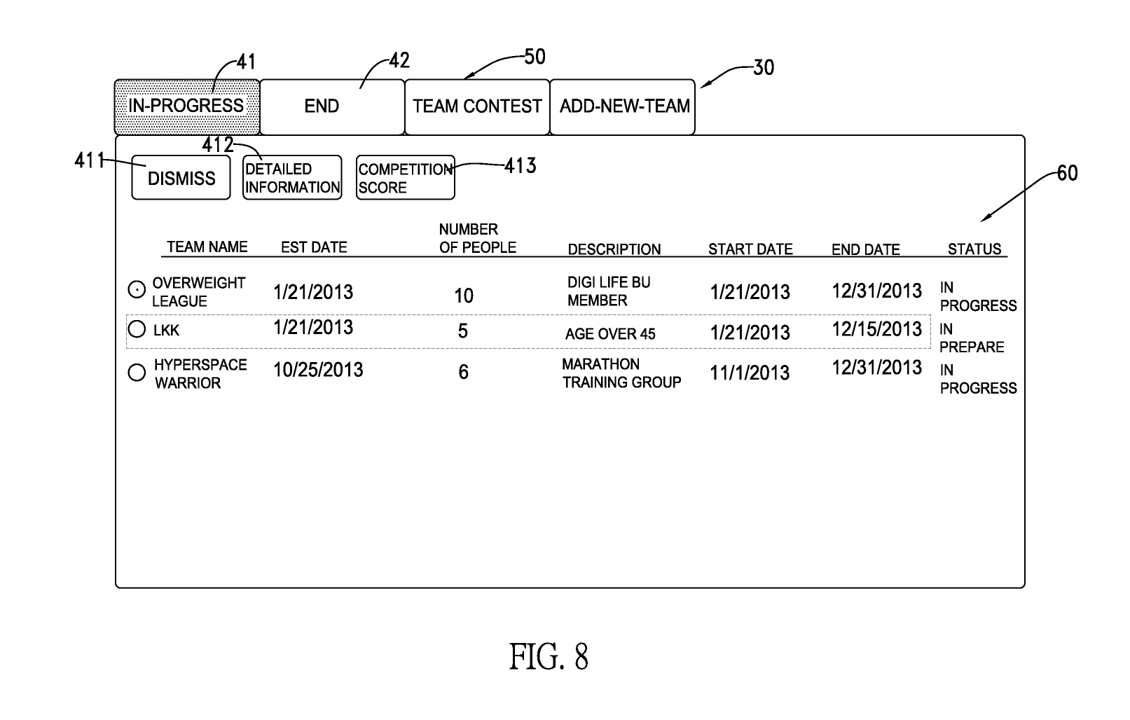

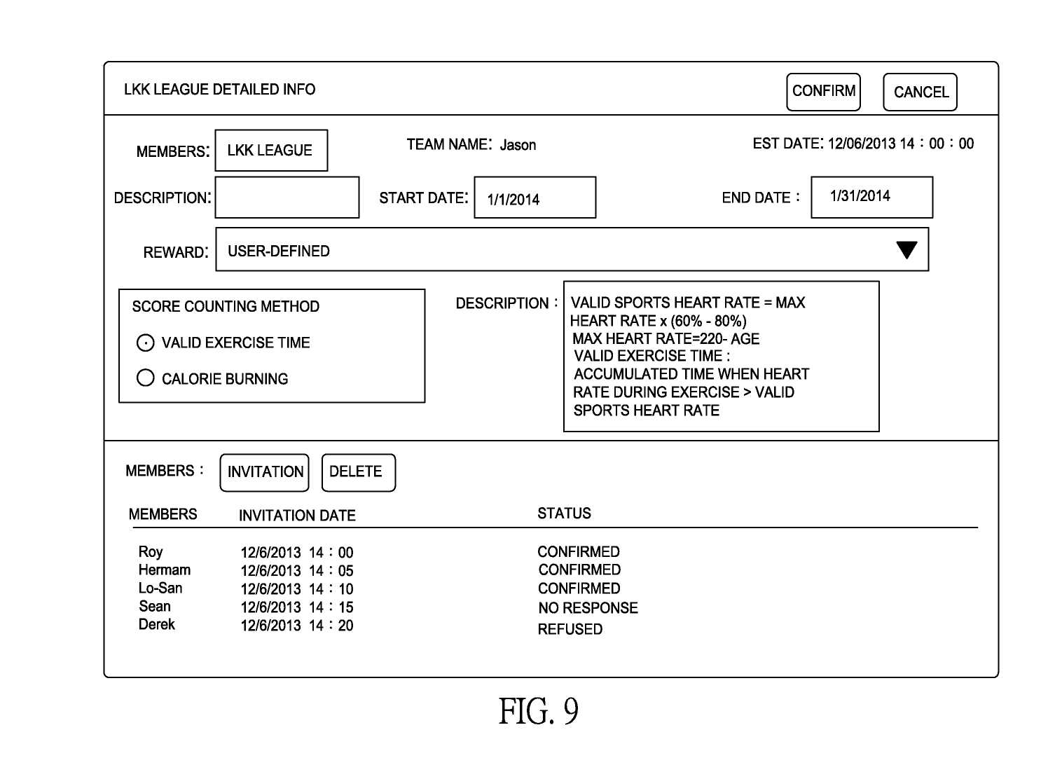

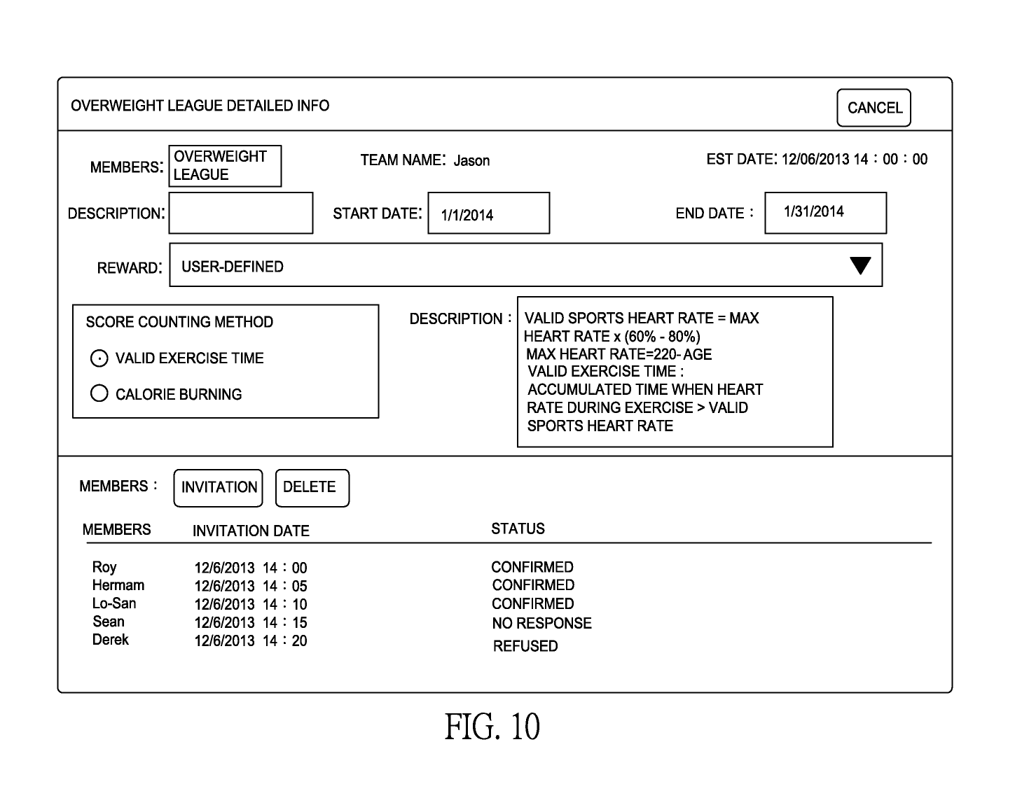

In the present embodiment, the in-progress menu 41 in the team-managing interface is clicked, with reference to FIG. 8, and a dismiss-function option 411, a detailed-information-function option 412, a competition-score-function option 413 are shown in the page. The detailed-information-function option 412 is to provide a detailed information page and a specification for one of the sports teams is editable. Before the competition is begun, the status column 60 shows “In preparation” and the team name, the competition start date, the competition end date, the reward method, the score counting mode still can be modified. With reference to FIG. 9, when the act to invite the members or remove the members is confirmed and the contest is started, any modifications are not allowed in the content. With reference to FIG. 10, only a cancelation option is provided to exit the present page, and any modifications are not allowed.

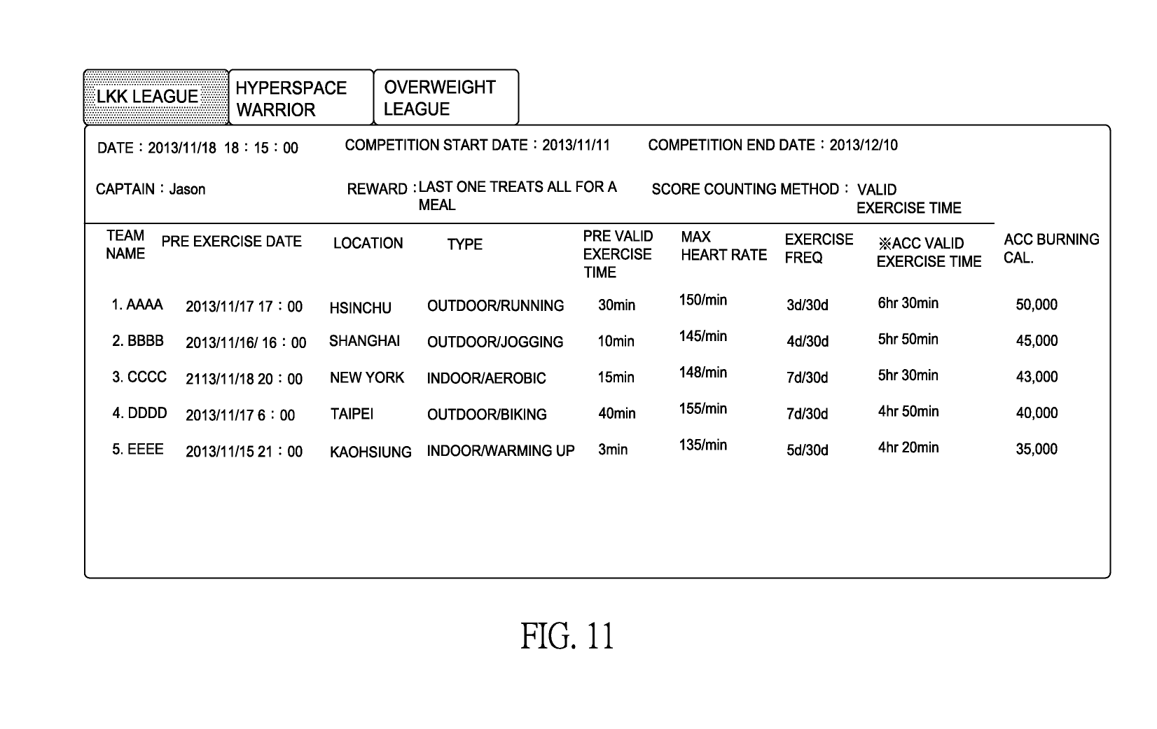

When the aforementioned sports team is established, the proposer (the captain) and the invitees (the members) can click the competition-score-function option 413 in the page with the in-progress menu 41 or the end menu 42 in the team-managing interface. The members can see the sports teams (such as a LKK league, a hyperspace warrior, an overweight league and so on) joined in a status column 60. When the name of the specific sports team (such as the LKK league) is clicked, a contest score page is immediately generated. With reference to FIG. 11, the sports scores for all of the members in the specific sports team are shown and the sports scores can be sorted in accordance with the valid exercise time or the calorie burning. Furthermore, the content of the contest score includes a member name, a previous sports time, a previous sports location, a previous sports type, a previous sports time, a maximum heart rate in previous sports time, a sports recurrence rate by now, a previous valid exercise time and a calorie burning accumulation value. The sequencing in accordance with the score counting mode (the valid sports score counting or calorie burning scoring) is selected by the captain before the contest. With reference to FIG. 11, if the sequence is the valid exercise time, a symbol “” is added in front of the column to represent that the rankings are shown in accordance with the column. In the contest score page, a login account of the user, who joins the sports team and is in competition, also shows as reference for searching.

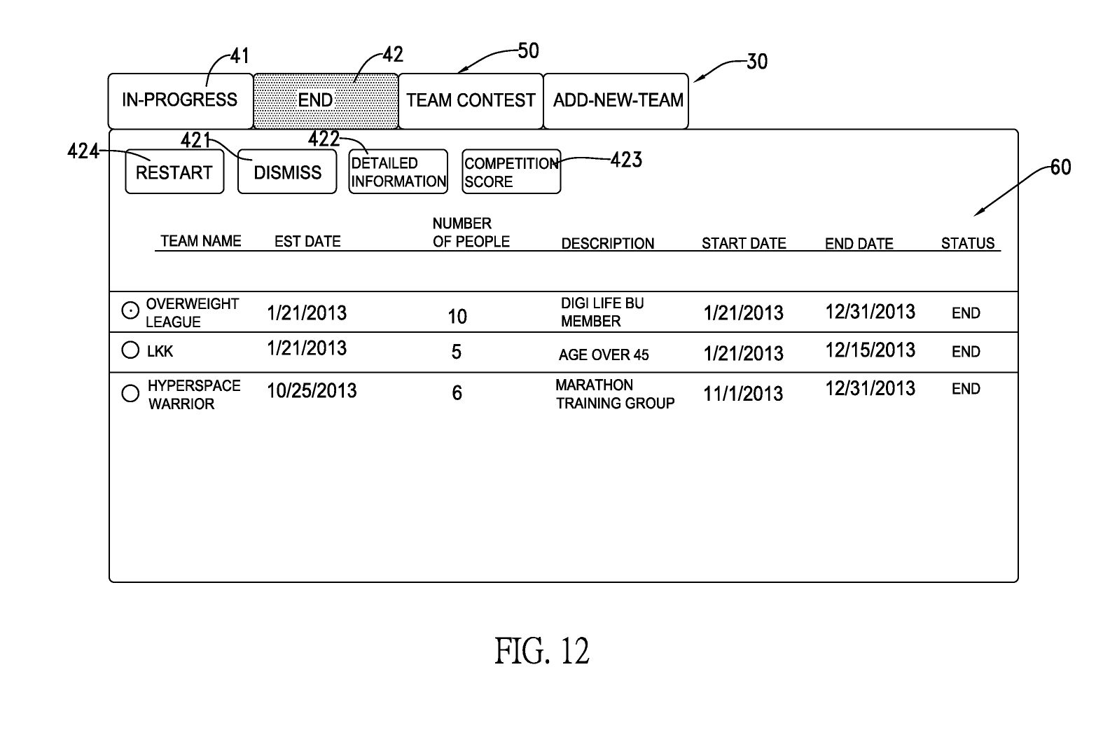

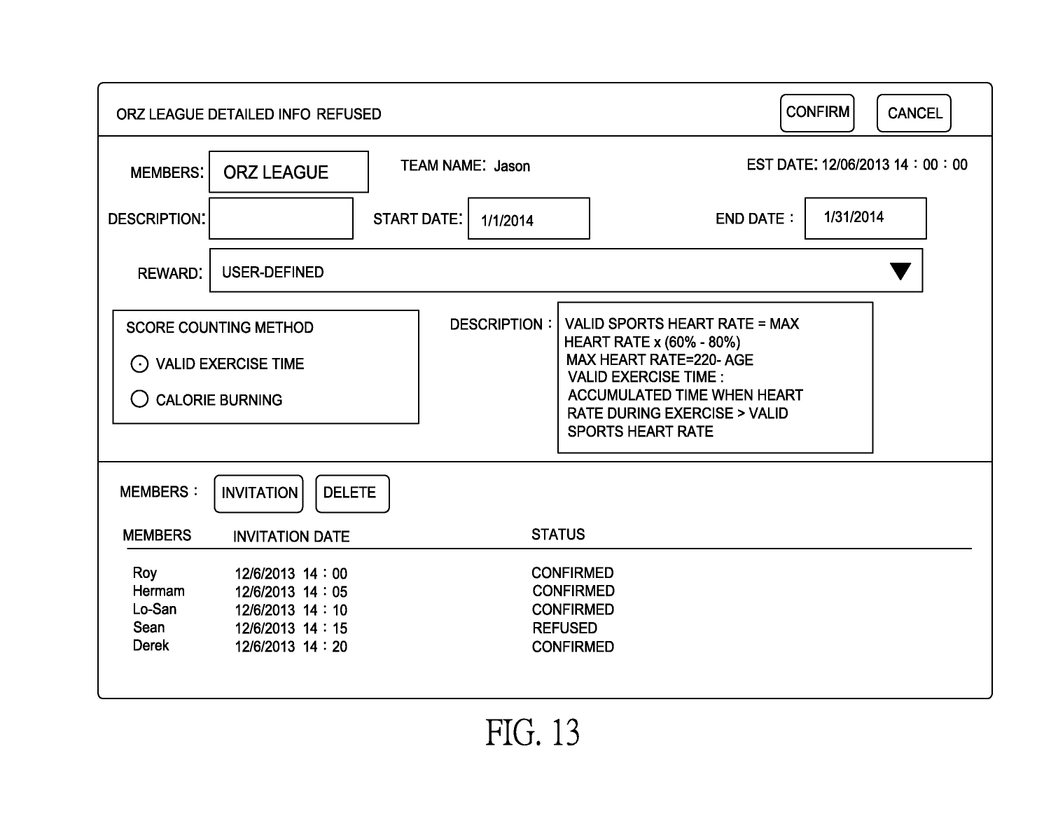

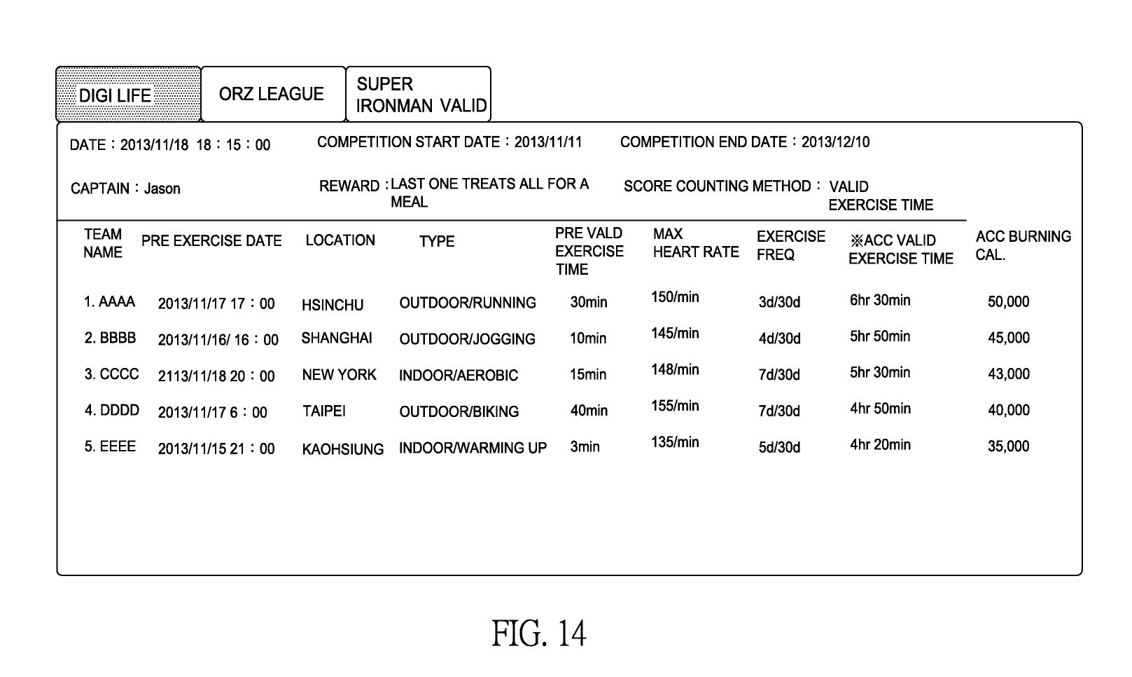

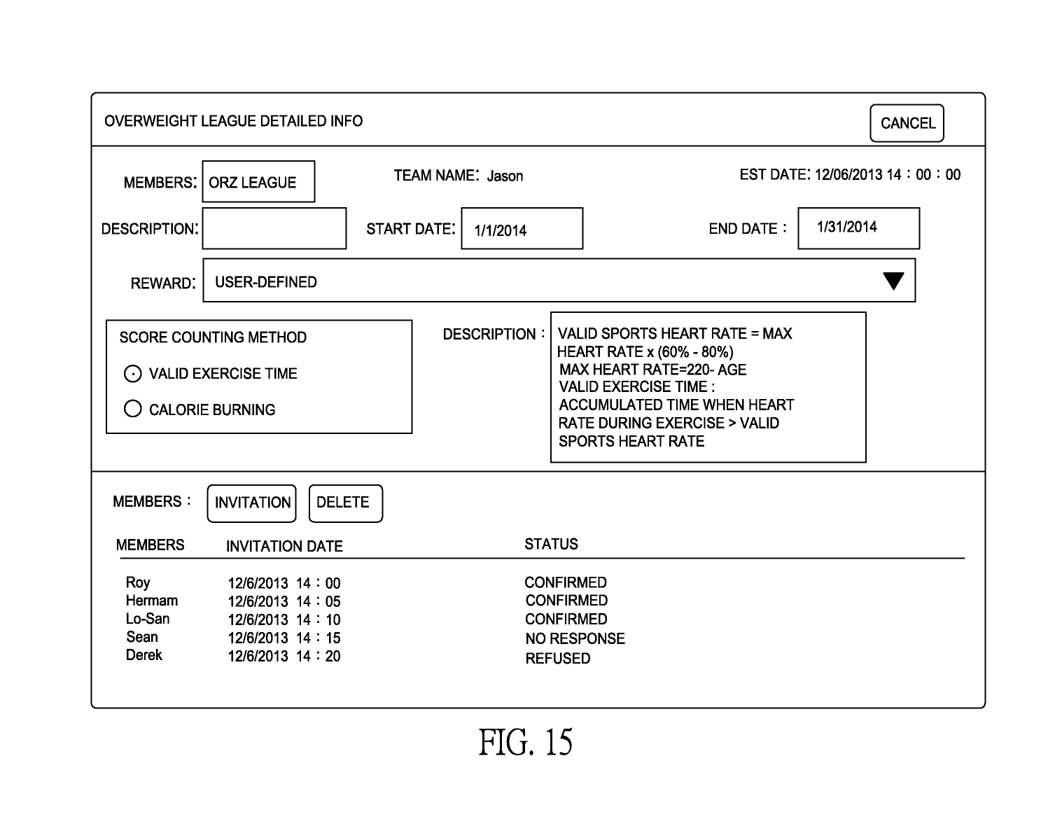

In the present embodiment, in addition to the ongoing competition, when the user wants to search for the ended competition, the end menu 42 in the team-managing interface can be clicked. With reference to FIG. 12, a dismiss-function option 421, a detailed-information-function option 422, and a competition-score-function option 423 are shown in the page. With reference to FIG. 13 and FIG. 14, the detailed-information-function option 422 provides a detailed information page with an end status. In addition, the page for the end menu 42 further includes a restart-function option 424. When the captain clicks the restart-function option 424, a restart page provides a competition customizing time, a scoring method, a reward method, a member invitation and so on for the captain to select. With reference to FIG. 15, when the captain clicks a confirmation option in the page, all of the invitation messages are sent to the rest of the members and the sports team is moved from the page of the end menu 42 to the page of the in-progress menu 41. If the captain clicks the dismiss-function option 421 and performs a confirmation option, the sports team is no longer shown in the status column 60. However, the information of the sports team is still stored in the cloud server 10 as reference for searching.

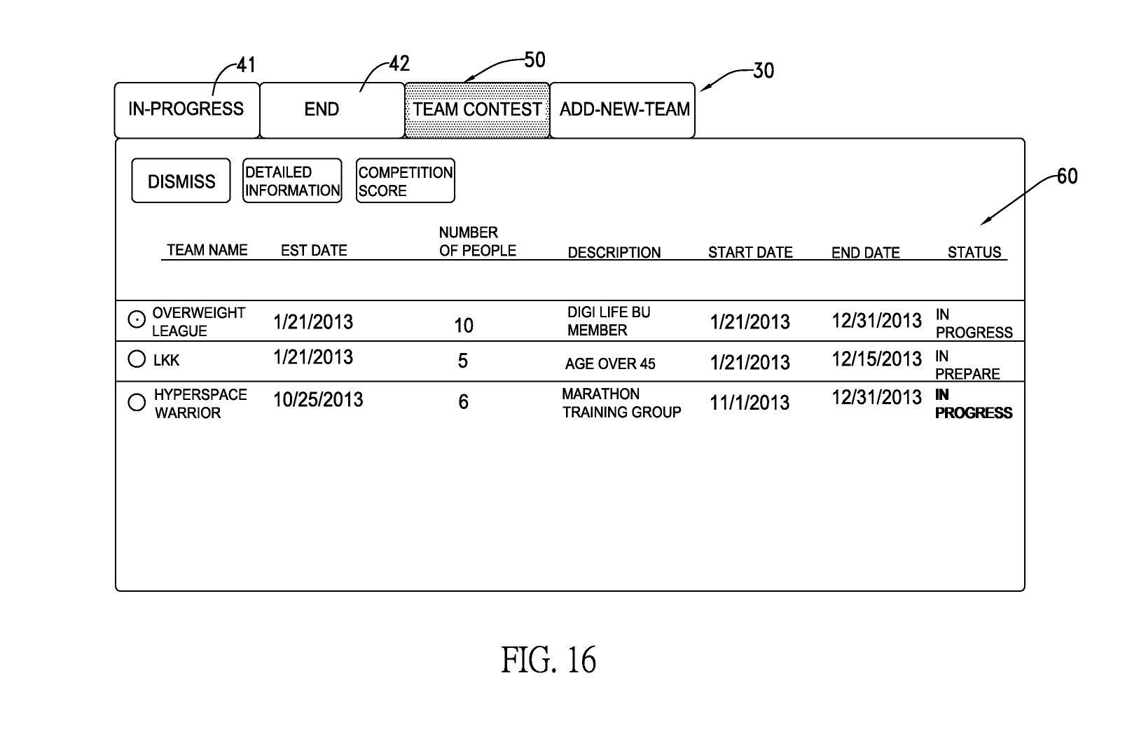

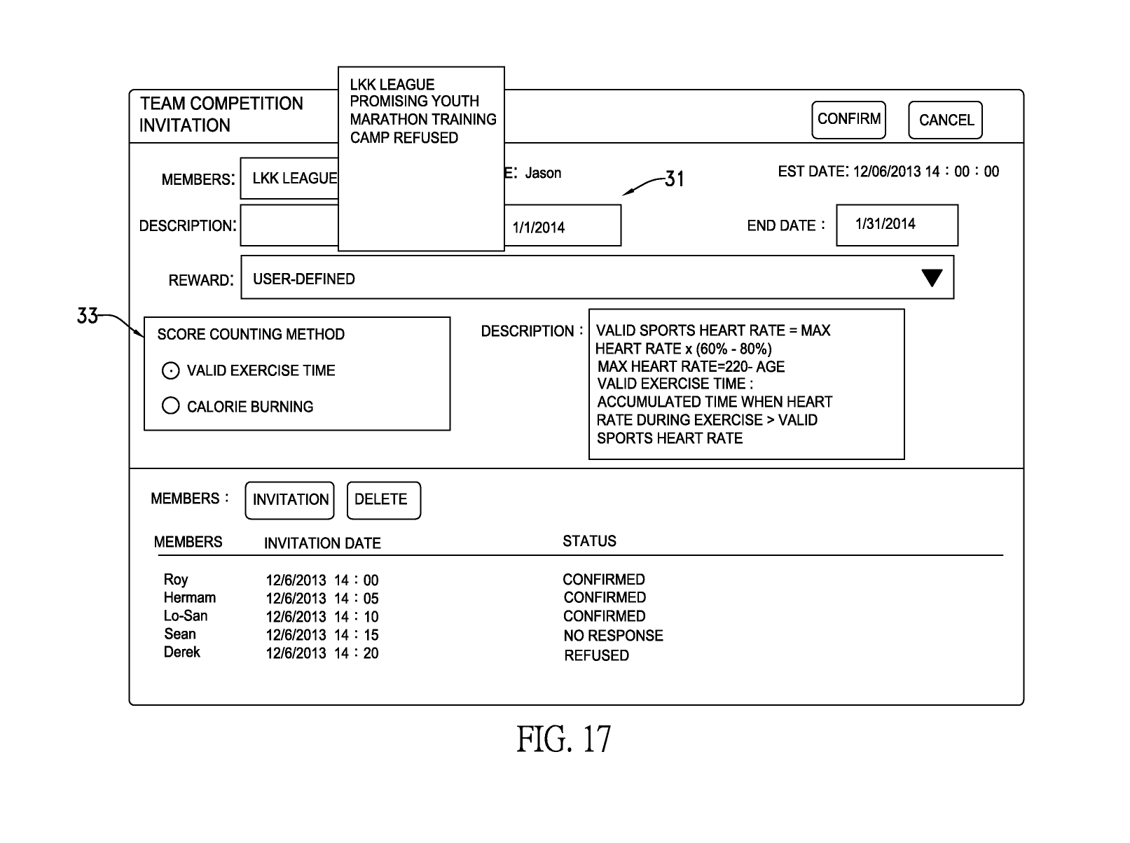

In order to specify that the captain can use the team-contest-customizing interface 50 in the present embodiment and how to make any one of the sports teams and more than one of the rest of the sports teams to join a team contest mode to perform at least one contest event and calculate the valid sports score of the sports team so as to encourage the users to increase the sports recurrence rate. With reference to FIG. 16, the page in the team-contest-customizing interface 50 includes a start-team-contest-function option 501, a detailed-information-function option 502, and a competition-score-function option 503. When the captain clicks the start-team-contest-function option 501, a team contest mode is provided for the captain to invite a captain in any one of the rest sports teams to join the contest. With reference to FIG. 17, the calculation of the competition score is based on the score counting mode developed in the score-counting-method option 33. When the score counting mode is the valid sport scoring, a valid exercise time spent by the member in the sports team is accumulated and a summation of the valid exercise time for all of the members is calculated. The valid sports scores for the sports teams in the competition event are sequenced. When the score counting mode is the calorie burning scoring, a calorie burning value generated by the members of the sports team is accumulated and a summation of the calorie burning values for all of the members is calculated. The calorie burning scores for the sports teams in the competition event are sequenced. The aforementioned objective is to let the members find time to exercise aggressively for team honor and the team score is getting better and better.

In the present embodiment, regarding the calculating method of the valid sports score, a wearable computing device with Internet function is carried by the members during the competition event. The wearable computing device will transmit the detected physiological value (such as heart rate) to the cloud server 10 for calculation. During the sports event, if the physiological value is greater than a valid sports heart rate, the time when the physiological value is greater than the valid sports heart rate is accumulated to be a valid sports score and the valid sports hear rate is a product that a maximum heart rate of a member times a certain percentage. The maximum heart rate is a difference between 220 and the age of the member. In the present embodiment, the valid percentage is between 55% and 100%.

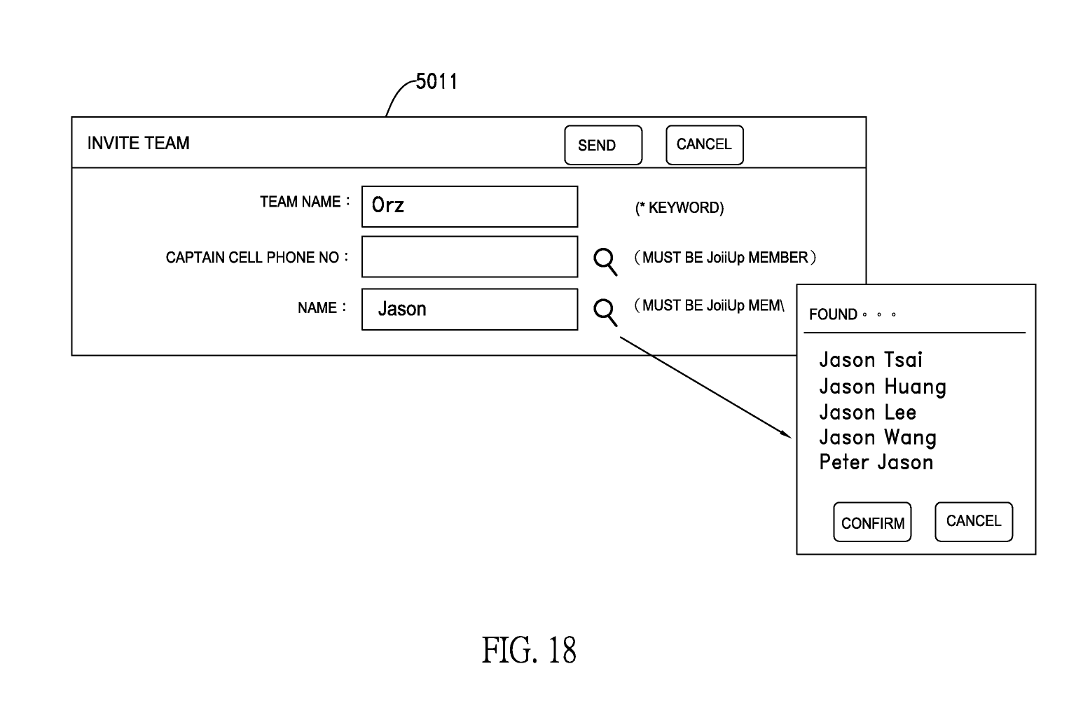

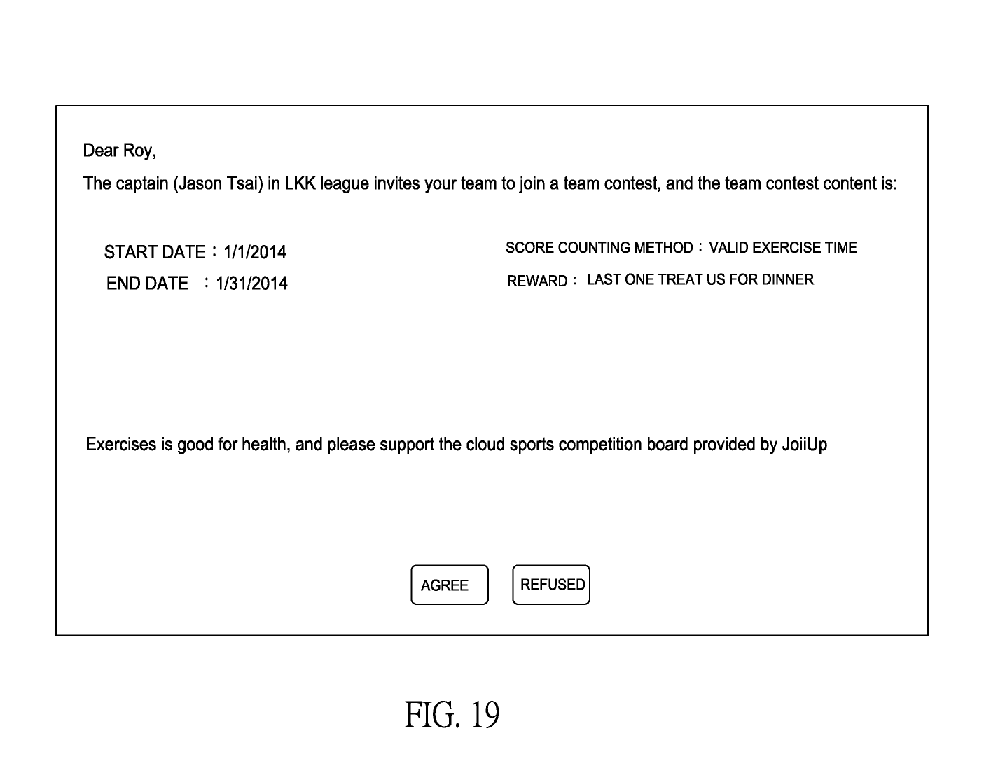

The captain initiating the aforementioned team contest mode can specify the competition time, the reward method and the scoring method, and search for and invite the sports teams when the captain recruits the members. The captain clicks the invitation option at the bottom of the team contest mode page to generate a team contest mode invitation page 5011. With reference to FIG. 18 and FIG. 19, the captain can enter the emails of the members to send the invitations or search for other users' names or cell phone numbers to send the invitations. After the invitations are sent, the sports information exchange platform will send contest mode invitation letters to the email boxes of the specific users.

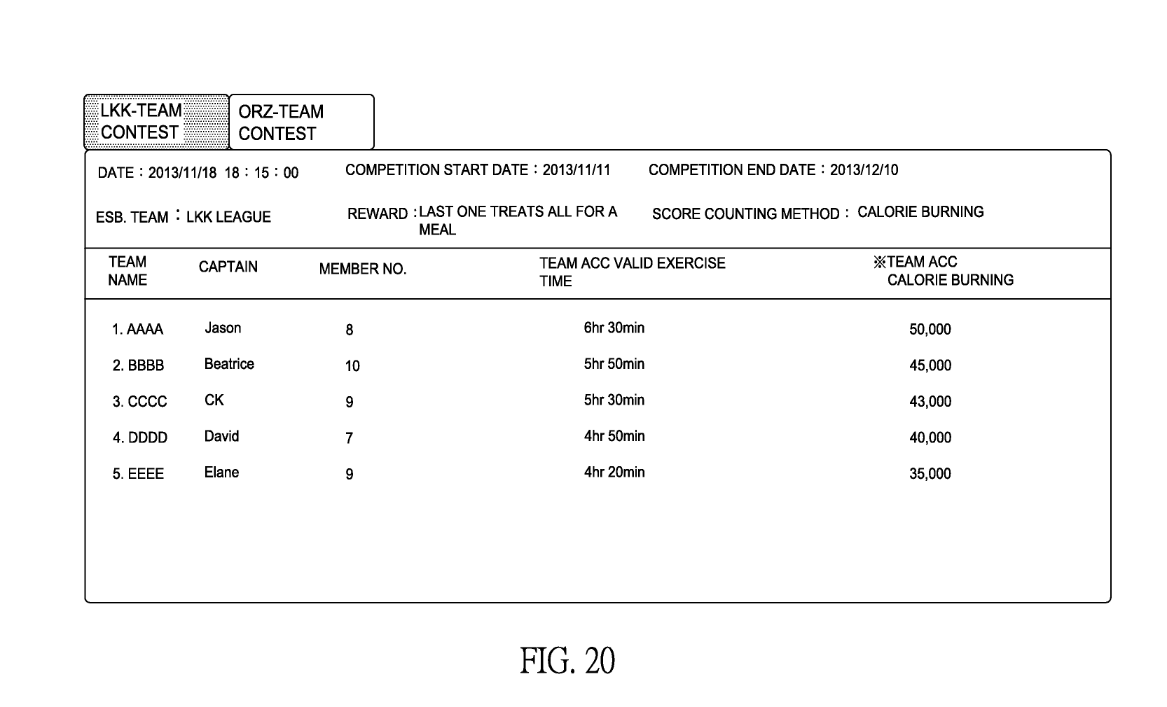

The contest score search is to click the competition-score-function option 503 to generate a contest score page for reference. With reference to FIG. 20, the contest scores of the sports teams can be seen in the contest score page and the joined sports teams (such as the LKK-contest) in the team contest mode are listed.

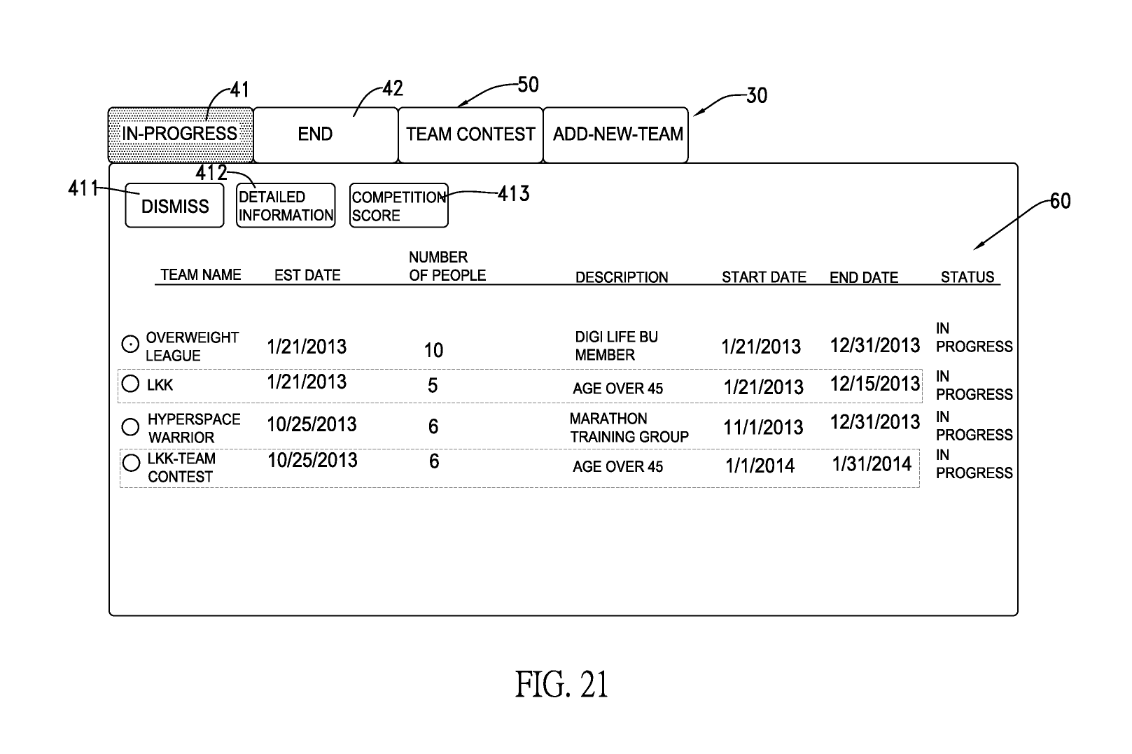

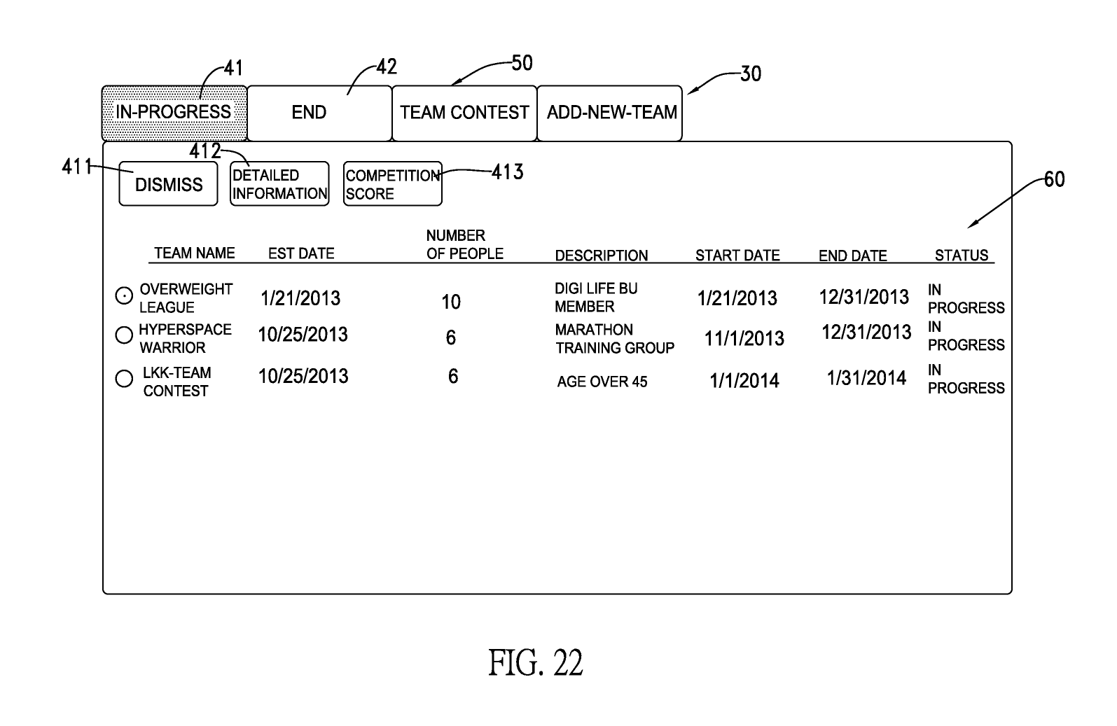

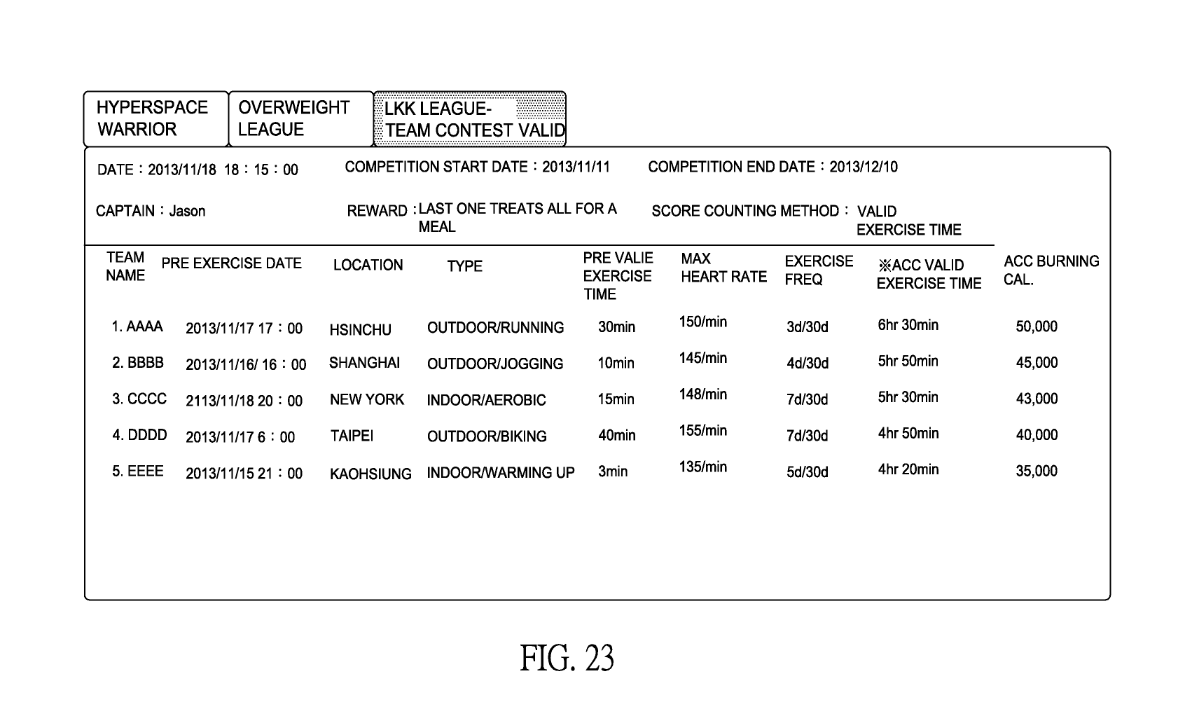

With reference to FIG. 21 and FIG. 22, when the sports team (LKK-team contest) is in the team contest mode and the sports team (LKK) is also in the competition period, the status column 60 shows the sports team (LKK) is in the competition and also shows the sports team (LKK-team contest) in the team contest mode is in progress. If the sports team (LKK) is not in the competition period but in the team contest mode, the sports team (LKK) is still shown in the page of the end menu 42. However, in the page of the in-progress menu 41, the sports team (LKK-team contest) under the team contest mode is shown in the page. Furthermore, with reference to FIG. 23, although the sports team (LKK) is in the team contest mode, the competition-score-function option 413 selected in the page of the in-progress menu 41 still can find out the current scores for all of the members in the competition score page of the competition-score-function option 413. The content of the competition score page includes the member name, the previous sports time, the previous sports location, the previous sports type, the valid exercise time in the previous exercise, the maximum heart rate in the previous exercise, the current sports recurrence rate, the accumulated valid exercise time and the accumulated burning calorie. The score is calculated based on the team contest mode of the competition period.

In the present embodiment, all of the users wear the wearable computing devices indoors or outdoors during exercise, and the sports record, the physiological value, etc will be uploaded to the cloud server 10 after the exercise is ended. The user has to use his account to log in, and the information regarding the sports recorder, the physiological value, etc will be recorded in the user account.

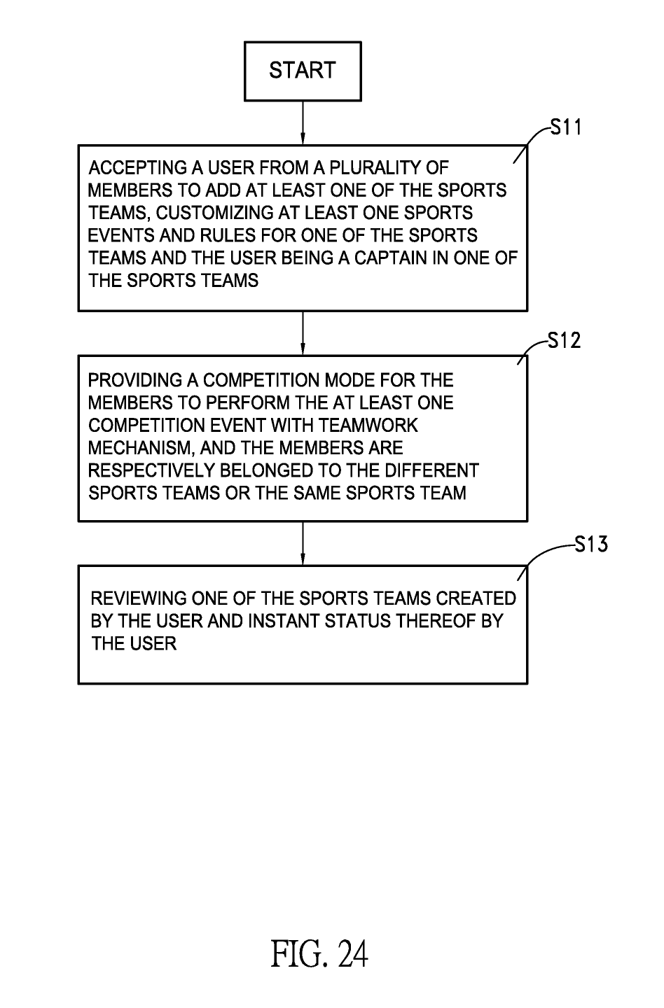

According to the specific application manner and description in the preferred embodiment of the present invention, an instant information exchange method for online sports teams can be summarized. The method is practiced in the cloud server 10 of the aforementioned sports information exchange platform. With reference to FIG. 24, the following steps are executed in the sports information exchange platform and include:

accepting a user from a plurality of members to create at least one of the sports teams, customizing at least one sports event and rules for one of the sports teams and the user being a captain in one of the sports teams (S11);

providing a competition mode for the members to perform the at least one competition event with teamwork mechanism (S12), and the members respectively belonging to the different sports teams or the same sports team;

reviewing one of the sports teams created by the user and instant status thereof by the user (S13).

According to the aforementioned method, the user is to become a proposer (the captain) in one of the sports teams and allows other users to join the sports team by inviting or requesting. The sports information exchange platform also provides a competition mode for the sports team and other sports teams to perform the competition events with teamwork mechanism. The members in the sports team have to work together and remind each other to finish the competition events. When the sports teams are in the competition events, the sports information exchange platform further allows the user to manage and review the sports teams and the instant status thereof at any time so as to enhance the efficiency of the sports team.

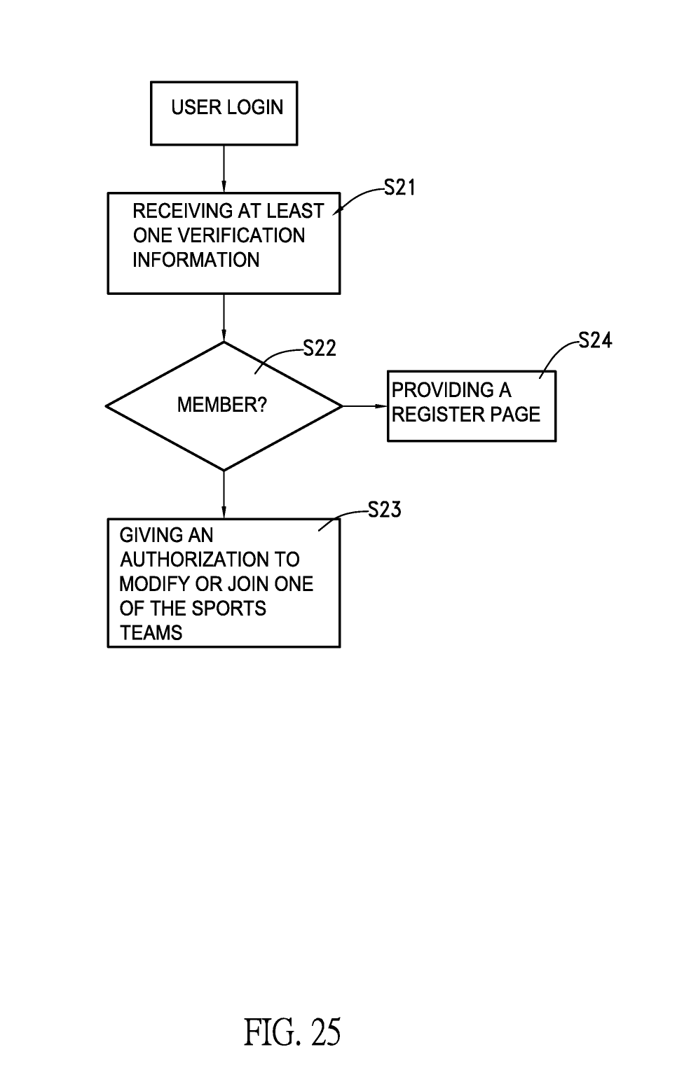

The present invention further provides a subscriber verification procedure and the user has to become a subscriber to log in the sports information exchange platform first. When the user links to the cloud server 10 by the aforementioned electronic device 20 with Internet function and logs in the sports information exchange platform, as shown in FIG. 25, the subscriber verification procedure includes the following steps:

logging in the sports information exchange platform by the electronic device 20 from the user and receiving at least one verification information inputted by the user in the cloud server 10 (S21);

determining whether the verification information is a subscriber information by the sports information exchange platform (S22);

giving an authorization to modify or join one of the sports teams if the verification information is the subscriber information (S23);

providing a register page when the verification information is not the subscriber information (S24), and the register page including multiple columns related to account information to edit the subscriber information.

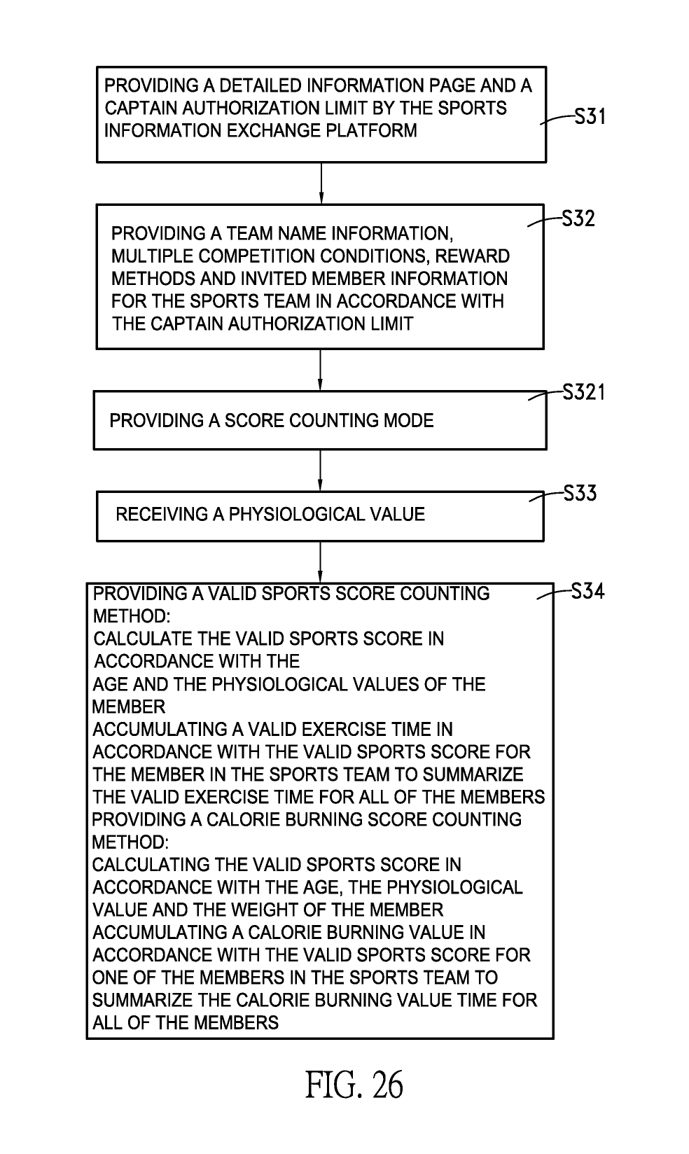

When the user becomes a subscriber with account authorization by the aforementioned subscriber verification procedure, the user can start to establish one of the sports teams and become the proposer (the captain) of the sports team. The present invention provides a procedure to customize the sports events and rules. When the aforementioned method performs the step of adding at least one of the sports teams, the procedure to customize the sports events and rules is executed. With reference to FIG. 26, the procedure further includes the following steps:

providing a detailed information page and a captain authorization limit by the sports information exchange platform (S31); and

providing a team name information, multiple competition conditions, reward methods and invited member information for the sports team in accordance with the captain authorization limit, and those data being editable (S32).

When the aforementioned method performs the step of providing a team name, multiple competition conditions, reward methods and invited member information for the sports team in accordance with the captain authorization limit, and those data is editable (S32), the method further includes a step of:

providing a score counting mode designed by the captain in the sports information exchange platform and a detailed description thereof (S321).

In order to describe the score counting mode provided in the present invention, the aforementioned method further includes the following steps:

receiving a physiological value provided by the electronic device 20 (S33), and the physiological value is heartbeat, heart rate, pulse, blood pressure, blood glucose, body temperature, electromyography (EMG), galvanic skin response (GSR), heart rate variability (HRV) or blood flow;

The sports information exchange platform provides a valid sports score counting method.

The valid sports score counting method is to calculate the valid sports score in accordance with the age and the physiological values of the member and accumulate a valid exercise time in accordance with the valid sports score for the member in the sports team to summarize the valid exercise time for all of the members. In addition, the valid sports score counting method also arranges a sequence for each one of the sports teams in the competition event.

The sports information exchange platform provides a calorie burning score counting method.

The calorie burning score counting method is to calculate the valid sports score in accordance with the age, the physiological value and the weight of the member and accumulate a calorie burning value in accordance with the valid sports score for one of the members in the sports team to summarize the calorie burning value for all of the members. In addition, the calorie burning score counting method also arranges the sequence for each one of the sports teams in the competition event (S34).

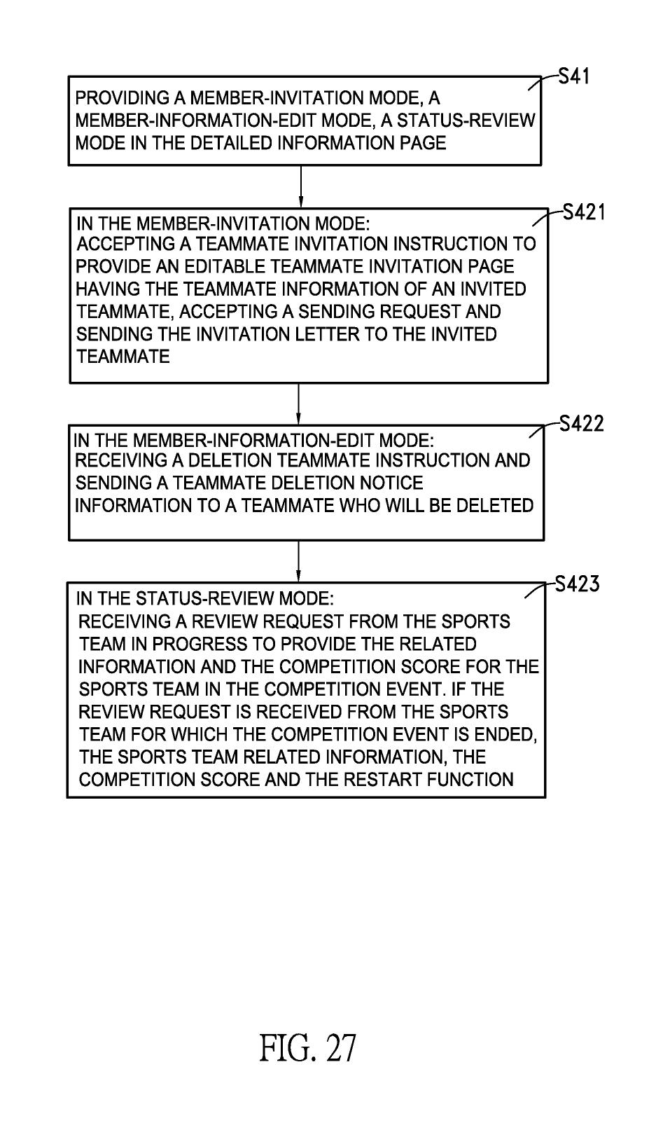

The present invention further includes an editing procedure for the data in the detailed information page. After the aforementioned method executes the step of providing a team name, multiple competition conditions, reward methods and invited member information of the sports team and those data is editable (S32), with reference to FIG. 27, the method further includes the following steps:

providing a member-invitation mode, a member-information-edit mode, a status-review mode in the detailed information page (S41) and the related information status also being provided;

In the member-invitation mode:

accepting a teammate invitation instruction to provide an editable teammate invitation page having the teammate information of an invited teammate, accepting a sending request and sending the invitation letter to the invited teammate (S421).

In the member-information-edit mode:

receiving a teammate deletion instruction and sending a teammate deletion notice information to a teammate who will be deleted (S422).

In the status-review mode:

receiving a review request from the sports team in progress to provide the related information and the competition score for the sports team in the competition event. If the review request is received from the sports team for which the competition event is ended, the sports team related information, the competition score and the restart function (S423) for the ended competition event is provided. If an execution instruction for the restart function is received, the method is going back to the step of establishing at least one of the sports teams (S11).

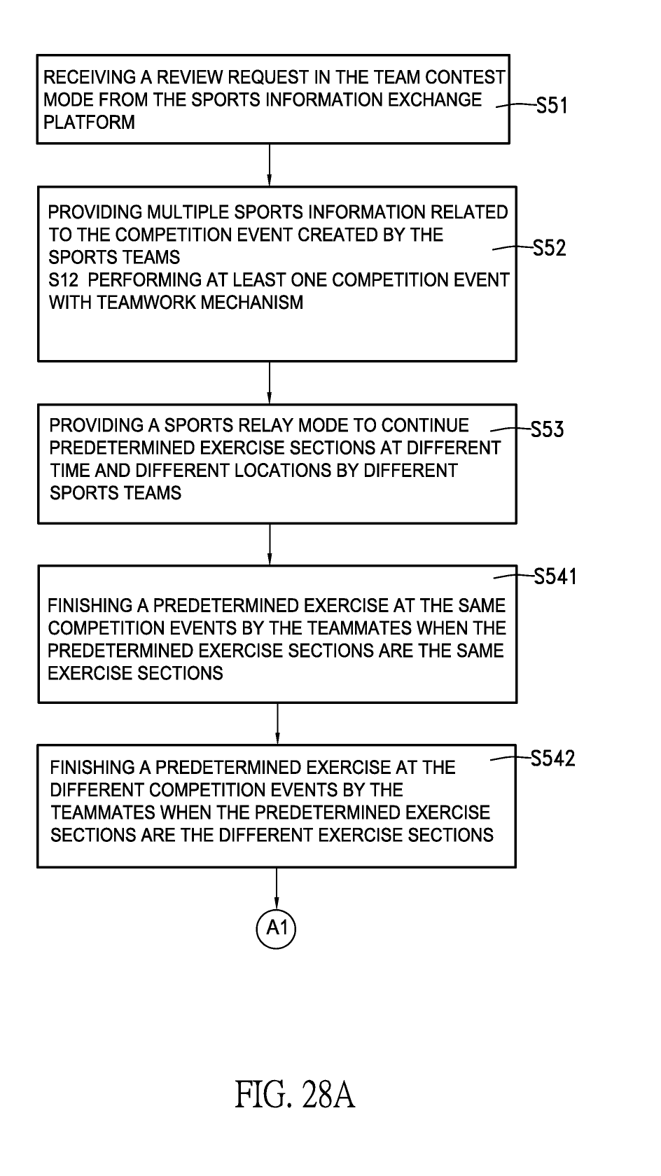

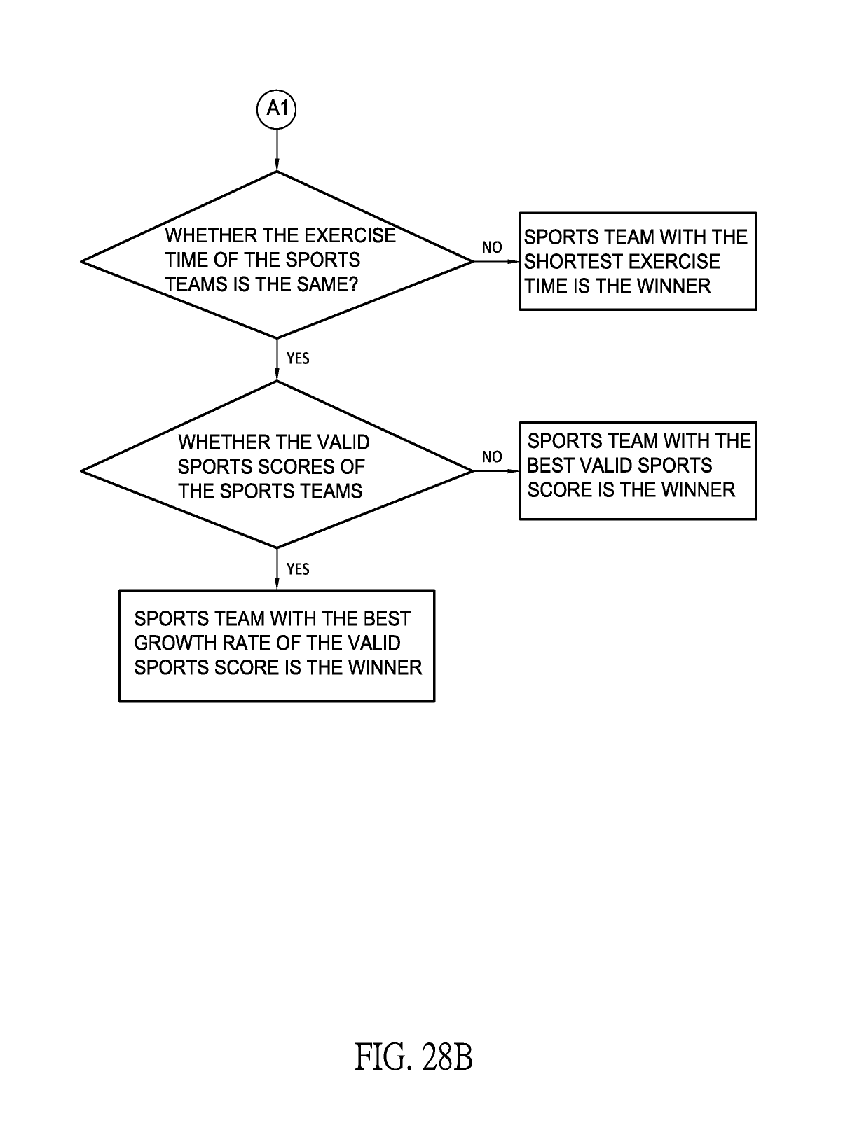

In order to describe a procedure to establish the competition mode and the teamwork mechanism provided in the present invention, with reference to FIG. 28, when the aforementioned method performs the step of providing a competition mode for the members to perform the at least one competition event with teamwork mechanism (S12), the procedure to establish the competition mode and the teamwork mechanism includes the following steps:

receiving a review request in the team contest mode from the sports information exchange platform (S51); and

providing multiple sports information related to the competition event created by the sports teams (S52);

When the step of performing at least one of the competition events with teamwork mechanism in the sports information exchange platform (S13) is executed:

a sports relay mode is provided in the sports information exchange platform to continue predetermined exercise sections at different time and different locations by different sports teams (S53).

When the predetermined exercise sections are the same exercise sections, the teammates finish a predetermined exercise at the same competition events (S541) and an exercise time for each of the sports teams is calculated.

When the predetermined exercise sections are the different exercise sections, the teammates finish a predetermined exercise at the different competition events (S542) and an exercise time for each of the sports teams is calculated.

determining whether the exercise time of the sports teams is the same (S55),

choosing the winner in the sports team with the shortest exercise time if the exercise time for each of the sports teams is different, (S551).

calculating the valid sports scores of the sports teams and determining whether the valid sports scores of the sports teams are the same if the exercise time of the sports teams is the same (S56); when the valid sports scores are different, the sports team with the best valid sports score is the winner (S561);

calculating a growth rate of the valid sports scores of the sports teams, and the sports team with the best growth rate of the valid sports score is the winner (S57).

In the aforementioned steps, the calculation method of the valid sports score is to accumulate the time when the heart rate of the teammate is greater than a valid sports heart rate. The calculation method of the growth rate of the valid sports score is to compare the new valid sports score of the sports team with the previous valid sports score. In addition, the competition event with teamwork mechanism includes a running event, a jogging event, a cycling event, an indoor aerobic event, a ball game, a swimming event and so on.

In summary, an online instant information exchange system for the sports team in another embodiment of the present invention implements the sports information exchange platform provided in the aforementioned cloud server 10 and the cloud server 10 is connected with the electronic device 20 via Internet for information exchange. The user from the members can add at least one of the sports teams in the sports information exchange platform by the electronic device 20. The proposer of the sports team can customize the competition events and the reward for the sports team and the teammates at the same sports team can perform the competition. The sports team can perform at least one of the sports events having teamwork mechanism together with other sports teams. The instant information exchange system can further receive the physiological values transmitted from the electronic devices 20 as reference for the competition score calculation. The sports team of the user can reduce the invitation time and the waiting time when the different sports teams are performing the competition so as to achieve the purpose of the exercise and the efficiency of the exercise. Regardless that the competition is between the teammates in the same sports team or between the different sports teams, the competition scores and results are automatically calculated and recorded in the sports information exchange platform. The inconvenience and mistakes caused by manual operation process can be reduced.

While the present invention has been described in terms of what are presently considered to be the most practical and preferred embodiments, it is to be understood that the present invention need not be restricted to the disclosed embodiment. On the contrary, it is intended to cover various modifications and similar arrangements included within the spirit and scope of the appended claims which are to be accorded with the broadest interpretation so as to encompass all such modifications and similar structures. Therefore, the above description and illustration should not be taken as limiting the scope of the present invention which is defined by the appended claims.

What is claimed is:

1. An instant information exchange method for online sports teams executed in a sports information exchange platform, and the method comprising steps of:
accepting a user from a plurality of members to create one of the sports teams;
customizing at least one sports event and rules in said one of the sports teams created by the user;
adding at least one of the members in said one of the sports teams created by the user;
providing a competition mode for the members to perform at least one competition event with teamwork mechanism; and
allowing the user to review said one of the sports teams created by the user and instant status in said one of the sports teams.

2. The instant information exchange method for online sports teams as claimed in claim 1, wherein the members belong to the same sports team during the competition mode.

3. The instant information exchange method for online sports teams as claimed in claim 1, wherein the members belong to the different sports teams during the competition mode.

4. The instant information exchange method for online sports teams as claimed in claim 1, wherein more than one of the sports teams are joined in the at least one competition event with teamwork mechanism during the competition mode.

5. The instant information exchange method for online sports teams as claimed in claim 1 further comprising a step of:
customizing a score counting mode with related instruction information.

6. The instant information exchange method for online sports teams as claimed in claim 1, wherein the step of allowing the user to review said one of the sports teams created by the user and instant status in said one of the sports teams further includes:
receiving a physiological value as reference for a competition score.

7. The instant information exchange method for online sports teams as claimed in claim 6, wherein the physiological value is heartbeat, heart rate, pulse, blood pressure, blood glucose, body temperature, electromyography (EMG), galvanic skin response (GSR), heart rate variability (HRV), or blood flow.

8. The instant information exchange method for online sports teams as claimed in claim 1 further comprising steps of:
providing a valid sports score counting method to covert ages of the members and the physiological value to be valid sports scores;
accumulating a valid exercise time in accordance with the valid sports scores of the members in one of the sports teams;
summarizing all of the valid exercise time for all of the members; and
arranging a sequence for each one of the sports teams in the competition event.

9. The instant information exchange method for online sports teams as claimed in claim 8, wherein a calculation method of the valid sports scores is to accumulate time when the physiological value in one of the members is greater than a valid sports heart rate to be the valid sports score.

10. The instant information exchange method for online sports teams as claimed in claim 9, wherein the valid sports heart rate is a product of a maximum heart rate in one of the members and a valid percentage.

11. The instant information exchange method for online sports teams as claimed in claim 10, wherein the maximum heart rate is a difference between 220 and the ages of the members of the sports teams, and a range of the valid percentage is from 55% to 100%.

12. The instant information exchange method for online sports teams as claimed in claim 1 further comprising steps of:
providing a calorie burning score counting method and calculating a valid sports score in accordance with the age, the physiological value and weight in one of the members;
accumulating a calorie burning value in accordance with the valid sports score of the members in the sports teams; and
summarizing the calorie burning value of the members and sequencing the sports teams in the competition event.

13. The instant information exchange method for online sports teams as claimed in claim 1, further comprising a step of:
providing an exercise relay mode to let the different sports teams do a plurality of predetermined exercise sections at different time and different locations.

14. The instant information exchange method for online sports teams as claimed in claim 13, wherein the predetermined exercise sections are different.

15. The instant information exchange method for online sports teams as claimed in claim 13, wherein the valid sports score in each one of the sports teams is used as reference to be a competition score when finishing time in the sports teams is the same.

16. The instant information exchange method for online sports teams as claimed in claim 13, wherein a growth rate of the valid sports score in each one of the sports teams is calculated as reference to be a competition score when finishing time in the sports teams is the same.

17. An instant information exchange system for online sports teams having a cloud server and the cloud server comprising:
an add-new-team interface configured to create one of the sports teams for a user from a plurality of members, customize a plurality of sports events and rules for said one of the sports teams created by the user, and create at least one of the members in said one of the sports teams created by the user;
a team-contest-customizing interface configured to let at least two of the members enter a competition mode and perform at least one competition event with teamwork mechanism; and
a team-managing interface configured to manage and review the sports teams and instant status of the sports teams for the user.

18. The instant information exchange system for online sports team as claimed in claim 17 further comprising at least one electronic device connected with the cloud server via Internet and each of the at least one electronic device including:
a network module;
a memory unit;
a display;
an input module; and
a processor connected with the network module, the memory unit, the display, and the input module;
wherein the at least one electronic device shows the add-new-team interface, the team-contest-customizing interface, and the team-managing interface on the display, operates from the input module by the user, and adds the new sports teams to perform the at least one competition event with teamwork mechanism.

19. The instant information exchange system for online sports team as claimed in claim 18, wherein each of the at least one electronic device includes a sensor, and the sensor is electrically connected with the processor and configured to detect a physiological value of the user and the physiological value is transmitted to the cloud server to be a reference for calculating a competition score.

20. The instant information exchange system for online sports team as claimed in claim 18, wherein each of the at least one electronic device includes a sensor, and the sensor is electrically connected with the processor and configured to detect a physiological value of the user and the physiological value is transmitted to the cloud server to calculate a valid sports score.

21. The instant information exchange system for online sports team as claimed in claim 19, wherein the physiological value is heartbeat, heart rate, pulse, blood pressure, blood glucose, body temperature, electromyography (EMG), galvanic skin response (GSR), heart rate variability (HRV), or blood flow.
